(19) United States
(12) Patent Application Publication
(10) Pub. No.: US 2023/0263864 A1
(43) Pub. Date: Aug. 24, 2023

(54) THERAPEUTIC MICROBES

(71) Applicant: Danmarks Tekniske Universitet, Kongens Lyngby (DK)

(72) Inventors: Mareike BONGERS, Kongens Lyngby (DK); Felipe TUEROS FARFAN, Kongens Lyngby (DK); Morten SOMMER, Kongens Lyngby (DK); Frederik NEERGAARD, Kongens Lyngby (DK); Susanne KAMMLER, Kongens Lyngby (DK)

(21) Appl. No.: 18/015,938

(22) PCT Filed: Jul. 15, 2021
(86) PCT No.: PCT/EP2021/069895

§ 371 Date: Jan. 12, 2023

(30) Foreign Application Priority Data
Jul. 15, 2020 (GB) 2010928.6
May 26, 2021 (GB) 2107473.7

(51) Int. Cl. A61K 38/44; C12N 15/52

(52) U.S. Cl. CPC A61K 38/44; C12Y 114/16002; C12Y 401/01028; C12Y 305/04016; C12Y 402/01096; C12N 15/52

(57) ABSTRACT

The invention relates to microbial cells and microbial cells for use as a medicament, the cells expressing a recombinant nucleic acid encoding a eukaryotic tyrosine hydroxylase. The cells produce L-DOPA and dopamine.

### FIELD OF THE INVENTION

The present invention relates to engineered microbes that are suitable for in vivo therapeutic production of L-DOPA and Dopamine in a subject. The invention also relates to pharmaceutical formulations and uses of the same for the treatment or management of diseases and disorders that can be treated or managed with L-DOPA and Dopamine produced by the engineered microbes in vivo in the digestive track, such as the gut.

### BACKGROUND

A number of bioactive molecules can be derived from L-tyrosine using different enzymes and enzymatic pathways as shown in FIG. 1. These include L-DOPA and dopamine, both of which have beneficial medicinal effects.

L-DOPA is a prodrug of dopamine that is administered to patients with Parkinson's due to its ability to cross the blood-brain barrier. Currently L-DOPA is administered as a pharmaceutical. However, maintaining a stable level of the compound in the blood is problematic.

Dopamine which is produced by decarboxylation of L-DOPA, modulates blood pressure, and also has a role in immune modulation, adipose tissue metabolism, nutrient absorption, and modulation of gut-brain axis functions.

### SUMMARY OF THE INVENTION

The present application addresses the need for these molecules by providing engineered microbes which can produce L-DOPA and dopamine in the gut.

In vivo production of compounds in the gut by engineered microbes is complicated by the nature of enzymatic processes and potential for a variety of by-products that complicates therapeutic in vivo use of the engineered microbes.

However, the present inventors have engineered microbes which produce L-DOPA and dopamine in sufficient amounts to have in vivo efficacy. The by-product profiles of the constructs are also suitable for therapeutic use.

In a first aspect, there is provided a microbial cell adapted to produce L-DOPA, the cell comprising: a recombinant nucleic acid encoding a eukaryotic tyrosine hydroxylase, for use as a medicament.

The microbial cell may for use in a method of treating Parkinson's disease or in a method of treating a dopamine-related disorder.

In a further aspect there is provided a microbial cell comprising a recombinant nucleic acid encoding a eukaryotic tyrosine hydroxylase, wherein the microbial cell is a therapeutic microbial cell, optionally E. coli Nissle.

In a further aspect there is provided a pharmaceutical formulation comprising a microbial cell wherein the microbial cell comprises a recombinant nucleic acid encoding a eukaryotic tyrosine hydroxylase.

The microbial cell may additionally comprise a nucleic acid encoding a compound which inhibits an L-DOPA metabolizing bacteria; or may be co-administered with:

i) a compound which inhibits an L-DOPA-metabolizing bacteria; or

ii) a further microbial cell which produces a compound which inhibits an L-DOPA-metabolizing bacteria.

The microbial cell may also additionally comprise:

a) a recombinant nucleic acid encoding a 4a-hydroxytetrahydrobiopterin dehydratase; and/or

b) an σ⁷⁰ promoter.

In a second aspect of the invention, there is provided a microbial cell adapted to produce dopamine, the cell comprising:

a) a recombinant nucleic acid encoding a eukaryotic tyrosine hydroxylase; and

b) a recombinant nucleic acid encoding an enzyme having L-DOPA decarboxylase activity.

The tyrosine hydroxylase be a mutant enzyme wherein:

a) the mutant tyrosine hydroxylase does not comprise a functional regulatory domain; and/or

b) the mutant tyrosine hydroxylase comprises a mutation in the catalytic domain.

The mutation may correspond to any one of amino acids 177-198 of SEQ ID NO. 2, optionally wherein the mutation is at an amino acid corresponding to amino acid 196 of SEQ ID NO. 2, optionally wherein the mutation is Ser196Glu or Ser196Leu; or any one of amino acids 22-43 of SEQ ID NO. 4, optionally wherein the mutation is at an amino acid corresponding to amino acid 41 of SEQ ID NO. 4, optionally wherein the mutation is Ser41Glu (SEQ ID NO. 6) or Ser41Leu (SEQ ID NO. 8).

The L-DOPA decarboxylase enzyme may belong to any one of the following:

a) EC:4.1.1.28, optionally wherein the enzyme has at least 70% sequence identity to SEQ ID NO.s 18, 20 or 22;

b) EC:4.1.1.105, optionally wherein the enzyme has at least 70% sequence identity to SEQ ID NO.s 20 or 22;

c) EC:4.1.1.25 optionally wherein the enzyme has at least 70% sequence identity to SEQ ID NO. 25.

Also provided is a pharmaceutical formulation comprising any of the microbial cells above which are adapted to produce dopamine.

Also provided is any of the microbial cells above adapted to produce dopamine for use as a medicament, for example in a method of treating a dopamine-related disorder.

The following may apply to any of the aspects above:

The tyrosine hydroxylase may belong to EC 1.14.16.2.

The tyrosine hydroxylase may not comprise the regulatory domain. For example, the tyrosine hydroxylase may comprise the catalytic domain and the tetramerization domain of the eukaryotic tyrosine hydroxylase enzyme, optionally wherein the tyrosine hydroxylase has at least 70% sequence identity to SEQ ID NO. 4.

The microbial cell may additionally comprise a nucleic acid encoding a mutant GTP cyclohydrolase I, the mutant GTP cyclohydrolase I having at least 70% sequence identity to SEQ ID NO. 10, and comprising one or more mutations wherein the mutant provides for an increased hydroxylation activity of the tyrosine hydroxylase. For example, the GTP cyclohydrolase I mutation may be at a position corresponding to amino acid 198 of SEQ ID NO. 10.

The microbial cell may further comprise a nucleic acid encoding a 4a-hydroxytetrahydrobiopterin dehydratase (phhB), optionally wherein the phhB belongs to EC 4.2.1.96 and/or has at least 70% sequence identity to SEQ ID NO. 14; and/or a nucleic acid encoding a dihydromonapterin reductase (FolM), optionally wherein the FolM has at least 70% sequence identity to SEQ ID NO. 12.

The nucleic acid(s) may be integrated into the genome of the microbial cell.

In a further aspect, there is provided a recombinant expression plasmid comprising:

a) a recombinant nucleic acid encoding a eukaryotic tyrosine hydroxylase; and any one or more of the following:

b) i) a recombinant nucleic acid encoding a 4a-hydroxytetrahydrobiopterin dehydratase; and/or
ii) an σ⁷⁰ promoter; and/or
iii) a recombinant nucleic acid encoding a compound which inhibits an L-DOPA metabolizing bacteria.

In a further aspect, there is provided a recombinant expression plasmid comprising:

a) a recombinant nucleic acid encoding a eukaryotic tyrosine hydroxylase; and

b) a recombinant nucleic acid encoding an enzyme having L-DOPA decarboxylase activity.

In a further aspect there is provided a mutant eukaryotic tyrosine hydroxylase wherein the mutation is at any one of amino acids 177-198 of SEQ ID NO. 2, optionally wherein the mutation is at an amino acid corresponding to amino acid 196 of SEQ ID NO. 2, optionally wherein the mutation is Ser196Glu or Ser196Leu. The tyrosine hydroxylase enzyme may also be the truncated form lacking the regulatory domain therefore, optionally the mutation is at any one of amino acids 22-43 of SEQ ID NO. 4, optionally wherein the mutation is at an amino acid corresponding to amino acid 41 of SEQ ID NO. 4, optionally wherein the mutation is Ser41Glu (SEQ ID NO. 6) or Ser41Leu (SEQ ID NO. 8).

### DESCRIPTION OF THE FIGURES

FIG. 1. Shows a schematic representation of the catecholamine biosynthetic pathway and tyrosine derived by-products of interest.

FIG. 2. Shows the conversion of L-tyrosine to L-DOPA. TyrR represses the transcription of several enzymes involved in the biosynthesis of tyrosine. Tyrosine hydroxylase (TH) uses tetrahydrobiopterin (BH4) as a cofactor for converting L-tyrosine into L-DOPA. FolE (T198I) has increased catalytic activity, increasing biosynthesis of tetrahydromonapterin.

FIG. 3. (A) Shows E. coli Nissle conversion of Tyrosine into L-DOPA, with or without a mutation in the folE gene on the genome, and expressing an optimized TyrH. E. coli Nissle strains were inoculated in biological triplicates and grown for 24 hours in M9 media with 0.4% glucose (Preculture). Production culture was inoculated in 1:100 ratio from the preculture and grown for 22 hours in M9 media with 0.4% glucose and supplemented with 100 mg/L of L-Tyrosine. Production cultures were centrifuged at 4500 RPMs and supernatant was collected for HPLC analysis. (B) Shows L-DOPA production of E. coli Nissle harbouring the folE mutation when supplementing different concentrations of L-Tyrosine into the production culture following the previously described method. (C) Shows L-DOPA production from different promoter constructs with no L-tyrosine supplemented.

FIG. 4 shows phhB expression increases L-DOPA production.

FIG. 5. (A) Shows L-DOPA production improvement by overexpressing part of the tetrahydromobipterin biosynthetic pathway from E. coli (FolE(T198I)) and FolM) and the pterin recycling enzyme (PhhB) from Chromobacterium violaceum. (B) Shows the biosynthetic pathway for tetrahydromonapterin in E. coli.

FIG. 6. (A) Shows the process by which L-DOPA is converted into dopamine and later m-tyramine in the intestine by microbial species. Standard treatment with carbidopa does not inhibit catalytic activity of microbial aromatic amino acid decarboxylases. (B) Left —Representation of L-DOPA AMT without co-expression of bacteriocins against E. faecalis, most of L-DOPA being produced by the AMT is turned into dopamine by E. faecalis metabolism. (B) Right—By co-expressing bacteriocins against E. faecalis, the AMT is able to deliver more L-DOPA since E. faecalis is inhibited in the vicinity of the AMT. This increases bioavailability of L-DOPA for the patient. FIG. 5. (C) Shows halos of inhibition in Brain Heart Infusion (BHI) media from E. faecalis surrounding L-DOPA producing E. coli Nissle spots and co-expressing bacteriocins (Hiracin JM79, ubericin A and Enterocin A). (D) Shows EcN producing L-DOPA can outcompete E. faecalis by expressing bacteriocins. (in A); EcN which produce bacteriocins produce more L-DOPA than a non-bacteriocin producer in a co-culture experiment with E. faecalis (in B); and EcN which produce bacteriocins delay the metabolism of tyrosine into tyramine by E. faecalis (C and D).

FIG. 7. Engineered, L-DOPA producing E. coli Nissle affects host metabolism in a mouse model. Strains of E. coli Nissle either with (‘EcN_DOPA’) or without (EcN_CTRL) the L-DOPA production genes were orally gavaged to mice, with or without additional IP treatment with carbidopa. Host responses were measured during 7 days after gavage. Metabolites from the dopaminergic and connected serotonergic pathways were quantified in plasma, and urine, and animal body weight was recorded. (A) Serotonin (5-hydroxytryptamine, 5-HT) concentrations in urine were decreased below detection level when treating mice with carbidopa, but were elevated with EcN_DOPA compared to the control strain (p=0.06, ANOVA). (B) Urine 5-hydroxytrptamine (5-HTP, the immediate precursor to serotonin) was slightly increased with EcN_DOPA compared to the control in the carbidopa treatment groups. (C) Plasma serotonin levels are also reduced by carbidopa treatment, and were increased (p=0.08, ANOVA) again with EcN_DOPA. (D) A reduction in animal body weight is seen after 7 days of treatment with EcN_DOPA, but not the control strain, in the absence of carbidopa treatment. N=8 animals per group. (E) Colony forming units (CFU) per grams of feces from mice treated with EcN_WT and EcN_DOPA, 2 days after a single gavage.

FIG. 8. Shows production of tyramine and dopamine from L-tyrosine and L-DOPA respectively by the action of an aromatic amino acid decarboxylase.

FIG. 9. Shows dopamine and different metabolites being produced by E. coli Nissle harbouring L-DOPA production plasmid (pHM181) and different aromatic amino acid decarboxylases (pMK-xx), when 100 mg/L tyrosine was added to the medium.

FIG. 10 shows L-DOPA production from various tyrH mutants.

FIG. 11. (A) Shows production of dopamine and other metabolites using a mutated tyrH (Ser196Leu/Glu), when 100 mg/L tyrosine was added. (B) Shows production of Dopamine and metabolites from pMUT based expression system.

FIG. 12. Plasmids used in the examples. Plasmids C-F are specifically designed for therapeutic in vivo production of L-DOPA and dopamine.

FIG. 13. L-DOPA production with different promoter and inclusion of different co-factors

FIG. 14. Integration of the constructs

FIG. 15. L-DOPA production comparison using different hydroxylases

FIG. 16. Validation of in vivo production

FIG. 17: Additional copy numbers of tyrosine hydroxylase show L-DOPA levels can be titrated for variable level delivery.

### DETAILED DESCRIPTION

### General Terms

Microbial Cell

By microbial cell is meant a bacteria and/or yeast cell.

The microbial cell may be a therapeutic cell meaning a cell suitable for use in medical treatment. These cells are nonpathogenic and may be commensal, i.e. part of the normal flora of the gut. The microbial cell may be an aerobic organism. Alternatively, the microbial cell may be an anaerobe which can survive and optionally grow in the presence of oxygen. That is, the microbial cell is not an obligate anaerobe. The microbial cell may be a probiotic microbial cell.

The microbial cell must be able to tolerate oxygen. That is, they can survive in the presence of oxygen. To test if a cell can survive in the presence of oxygen, this can be done for instance using the thioglycolate test. Fluid thioglycolate media is made such that an oxygen gradient concentrates high oxygen at the top of the broth and low oxygen at the bottom of the broth. Organisms that tolerate oxygen will cluster near the top and organisms that cannot tolerate oxygen will cluster near the bottom.

Microbial cells which are anaerobes and can survive in the presence of oxygen are as follows: The microbial cell may be a facultative anaerobe. A facultative anaerobe can grow without oxygen but can use oxygen if present. Alternatively, the microbial cell may be an aerotolerant anaerobe which cannot use oxygen for growth but will tolerate it's presence.

The microbial cell may be able to colonize where there is oxygen in the small and/or large intestine, for example an oxygen gradient. For example, the mucous layer of the small and/or large intestine, for example the inner and/or outer layer of mucous. For example the inner or outer layer of mucous of the large intestine.

Suitable therapeutic cells include Escherichia coli, for example E. coli Nissle. Other examples of suitable therapeutic cells include lactic acid bacteria for example Lactobacillus and/or Lactococcus. Other examples of therapeutic cells include Akkermansia, for example Akkermansia muciniphila, Bifidobacterium, Bacteroides, Salmonella or Listeria.

Other examples include Saccharomyces boulardii.

The cell may alternatively be a synthetic microbial cell.

Where the microbial cell is a combination of cells, the yeast may for example produce tyrosine hydroxylase and optionally any 1 or more of the co-factors: FoIE, FolM, FoIX or phhB; and the bacterial cell may produce any 1 or more of the co-factors: FoIE, FolM, FoIX or phhB. For example, the yeast cell may produce tyrosine hydroylase and the bacterial cell may produce FolE and FolM.

The microbial cell may be a combination of bacterial cells also where one type of bacterial cell produces tyrosine hydroxylase and optionally 1 or more of the co-factors, and another type of bacterial cell produces one or more of the co-factors.

The resulting combination of microbial cells may be described as a composition of microbial cells.

Mutant

By mutant is meant an enzyme which differs from the full length wild-type form.

By corresponding to is meant the equivalent amino acid in any sequence for that enzyme. For example Ser 196 in a tyrosine hydroxylase other than rat. The corresponding or equivalent amino acid in a tyrosine hydroxylase from another species can be found using sequence alignment software such as the BLAST sequence alignment tool described below.

Nucleic Acids

The nucleic acids may have 70, 75, 80, 85, 90, 95 or 100% sequence identity with those listed in Table 3.

Pharmaceutical Formulation

A pharmaceutical formulation includes excipients to preserve the activity or to deliver the cell to the gut. Preferably the formulation is an oral formulation.

The microbial cell may be formulated to preserve its activity and/or for delivery to the gut via an oral tablet or capsule or the like.

For example, the microbial cell may be lyophilized and include a lyoprotectant. The formulation may alternatively or additionally include any other excipient required to preserve the activity of the cell.

The formulation may be in an oral dosage form with a coating which allows delivery to the gut, for example an enteric coating.

Plasmid

The enzymes for expression in the microbial cell may be cloned into one of the native plasmids of a therapeutic bacteria.

For example, E. coli contains 2 native plasmids which are maintained stably in the strain. Cloning the enzymes into these plasmids ensures stability of the plasmid and enzymes at a controlled, low copy number. Additionally, this minimizes the amount of foreign DNA introduced to the strain, and it is non-transferrable to other bacteria, ensuring safety.

Alternatively, the enzymes may be expressed in a plasmid which is not native to the bacteria.

A yeast plasmid may also be used when yeast is the or one of the microbial cell(s). The plasmid may comprise any of the enzymes and/or promoters listed below in combination for expression of L-DOPA or dopamine in the microbial cell.

Integrated into the Genome

Alternatively, the genes encoding the enzymes may be integrated into the genome of the therapeutic microbial cell. This can be done using the CRISPR technique. Alternatively this can be done by various other methods including clonetegration (Shearwin et al (2013), ACS Synthetic Biology, Vol 2, pp 537-541).

Promoters

A promoter is a nucleotide sequence capable of controlling the expression of a gene. The promoter may be a σ⁷⁰ promoter or a modified version of such a promoter where the nucleotide composition has been optimized for in vivo expression levels.

The promoters claimed have been tested for predictability and robustness in the mammalian GI tract. They have been selected from a large library of promoters, causing the most stable gene expression under any conditions (e.g. +/−oxygen, in exponential or stationary growth phase, in the upper and lower part of the GI tract, in the lumen vs. in the mucus layer), which are important for making robust therapeutic bacteria.

The tyrosine hydroxylase and/or L-DOPA decarboxylase genes may be under the control of the promoter. Additionally one or more of the other enzymes for L-DOPA or dopamine production listed may also be under the control of the promoter. Therefore, the microbial cell or recombinant plasmid may comprise one or more of the following promoters.

The σ⁷⁰ promoter may have at least 70, 75, 80, 85, 90, 95 or 100% sequence identity to SEQ ID NO. 32 or 33.

For example, the promoter for the tyrosine hydroxylase may have a consensus sequence as follows:

(SEQ ID NO. 55)
DSNYKNRYDMDHBRNDHYBVHNHNBNDDDDNHKDNN

Where the sequence is in accordance with the IUPAC code below.

| IUPAC nucleotide code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| . or — | gap |

For example, the promoter may be SEQ ID NO. 32 or 33 or a sequence comprising 90, 95 or 98% sequence identity with either SEQ ID NO. 32 or 33. The promoter may consist of consensus sequence SEQ ID NO. 55.

The promoter for any or all of FoIE, FolM and/or FoIX may be an Anderson promoter. The promoter for any or all of FoIE, FolM and/or FoIX may have a consensus sequence as follows (again with reference to the IUPAC code above):

(SEQ ID NO. 56)
YTKAYRGCTAGCTCAGYCCTWGGKAYWRTGCTAGC.

For example, the promoter may be SEQ ID NO. 38-50 or a sequence comprising 70, 75, 80, 85, 90, 95 or 98% sequence identity with either SEQ ID NO. 38-50. The promoter may consist of consensus sequence SEQ ID NO. 56.

Functional variants with different degrees of sequence identity can be checked for retention of activity by comparing expression of a suitable reporter under the control of the variant promoter and compare this activity with the reporter under the control of the non-variant promoter. It is generally preferred that a promoter with less that 100% sequence identity retains at least 25, 50, 75, 80, 85, 90, 95 or 100% activity of the reference promoter. In addition to sequence identity, the promoters may be shortened at 1 or both ends of the sequence. This shortening may be by 1 or 2 nucleotides at 1 or both ends. These shortened variants can be checked for retention of activity as explained above.

Recombinant

By recombinant is meant an exogenous nucleic acid sequence which is not native to the cell in which the nucleic acid is being expressed.

The cell may contain 1 copy of the enzyme(s) or more than 1. For example, there may be more than 1 copy of the nucleic acid encoding the tyrosine hydrolase present in the cell, either in a plasmid or integrated into the genome.

Sequence Identity

Sequence identity may be calculated using any suitable software such as BLAST (Altschul, S. F., Gish, W., Miller, W., Myers, E. W. & Lipman, D. J. (1990) “Basic local alignment search tool.” J. Mol. Biol. 215:403-410.)

The enzymes claimed may have at least 70%, 75%, 80%, 85%, 90%, 95% or 90% sequence identity to any of the enzymes listed in Table 3. The enzymes may additionally be truncated to the core secondary structure elements to provide function, for example by removing 1 to 20 (for example 1, 2, 3, 4, 5, 6, 7, 8, 9, 10, 11, 12, 13, 14, 15, 16, 17, 18, 19, 20) amino acids from the N and/or C termini of the construct.

Features for L-DOPA Production

L-DOPA

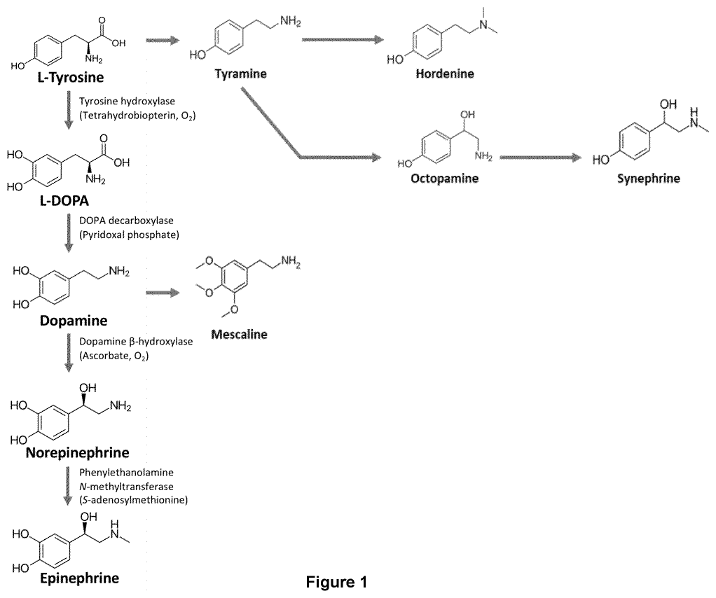

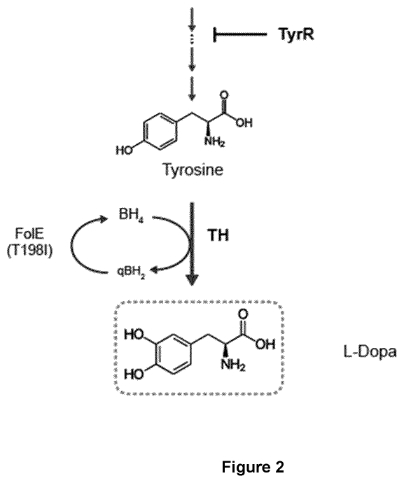

L-DOPA, L-3,4-dihydroxyphenylalanine, is made from the amino acid tyrosine. This is shown in FIGS. 1 and 2 along with the structure of L-DOPA.

It is the precursor to the neurotransmitter dopamine. Conversion to dopamine occurs in the CNS (after L-DOPA crosses the blood brain barrier) and in the peripheral nervous system.

Tyrosine Hydroxylase

The eukaryotic tyrosine hydroxylase (TyrOH) is a member of the biopterin-dependent aromatic amino acid hydroxylase family of non-heme, iron(II)-dependent enzymes. TyrOH catalyzes the conversion of tyrosine to L-dihydroxyphenylalanine (L-DOPA) as shown in FIG. 2.

The tyrosine hydroxylase of the invention may belong to EC 1.14.16.2. The enzyme may be an animal enzyme, for example a mammalian enzyme.

The sequence of the full length rat tyrosine hydroxylase is as follows:

MPTPSAPSPQPKGFRRAVSEQDAKQAEAVTSPRFIGRRQSLIEDARKER
EAAAAAAAAAVASSEPGNPLEAVVFEERDGNAVLNLLFSLRGTKPSSLS
RAVKVFETFEAKIHHLETRPAQRPLAGSPHLEYFVRFEVPSGDLAALLS
SVRRVSDDVRSAREDKVPWFPRKVSELDKCHHLVTKFDPDLDLDHPGFS
DQVYRQRRKLIAEIAFQYKHGEPIPHVEYTAEEIATWKEVYVTLKGLYA
THACREHLEGFQLLERYCGYREDSIPQLEDVSRFLKERTGFQLRPVAGL
LSARDFLASLAFRVFQCTQYIRHASSPMHSPEPDCCHELLGHVPMLADR
TFAQFSQDIGLASLGASDEEIEKLSTVYWFTVEFGLCKQNGELKAYGAG
LLSSYGELLHSLSEEPEVRAFDPDTAAVQPYQDQTYQPVYFVSESFNDA
KDKLRNYASRIQRPFSVKFDPYTLAIDVLDSPHTIQRSLEGVQDELHTL
AHALSAIS

The above sequence is SEQ ID NO. 2. The tyrosine hydroxylase may have at least 70, 75, 80, 85, 90, 95, 97 or 100% sequence identity with SEQ ID NO. 2.

The enzyme may be truncated to the core secondary structure elements to provide function, for example by removing 1 to 20 (for example 1, 2, 3, 4, 5, 6, 7, 8, 9, 10, 11, 12, 13, 14, 15, 16, 17, 18, 19, 20) amino acids from the N and/or C termini of the construct.

GTP Cyclohydrolase I (folE)

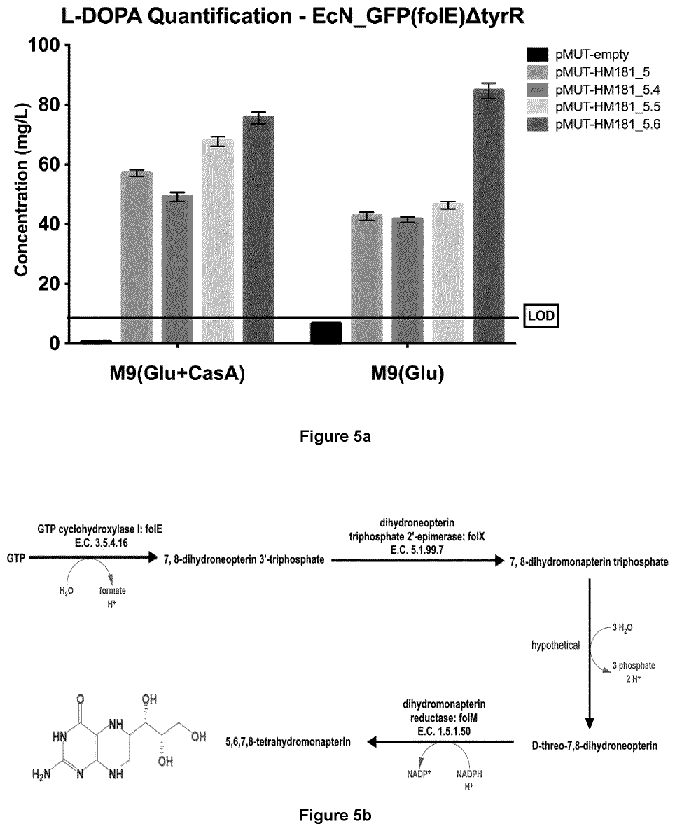

In humans, the production of L-DOPA requires synthesis and regeneration of the co-factor tetrahydrobiopterin. Bacteria and yeast do not produce this co-factor. Therefore, the native cofactor tetrahydromonapterin pathway is exploited instead. The synthesis pathway for this native cofactor is shown in FIG. 5b. GTP cyclohydolase I is an enzyme in this synthesis pathway.

The GTP cyclohydrolase I may belong to E.C. 3.5.4.16.

The GTP cyclohydrolase I may have at least 70, 75, 80, 85, 90, 95 or 100% sequence identity with SEQ ID NO. 10. The enzyme may additionally be truncated to the core secondary structure elements to provide function, for example by removing 1 to 20 (for example 1, 2, 3, 4, 5, 6, 7, 8, 9, 10, 11, 12, 13, 14, 15, 16, 17, 18, 19, 20) amino acids from the N and/or C termini of the construct.

The mutation may increase hydroxylation of the tyrosine hydroxylase by at least 120% as compared to the native or wild-type unmutated enzyme (under the same conditions). The mutation may be at any one of the following positions in SEQ ID NO. 10: D97-E112, K121-D130, N170-H180, S193-L200 and S207-N222. For example, D97, M99, T101, V102, A125, K129, N170, V179, T196, T198 (excluding T198P), S199, L200, S207, H212, E213, F214, L215 and H221.

The mutation may be selected from: D97V, D97L, D97A, D97T, M99C, M99T, M99V, M99L, M991, T101I, T101V, T101L, V102M, N170K, N170D, N170L, V179A, V179M, T1961, T196V, T196L, T198I, T198V, T1983, T198L, 3199Y, 3199F, L200P, L200C, L2003, L200A, S207R, S207K, S207M, H212R, H212K, E213K, E213R, F214A, F214G, F2143, L215P, L215Q, L215N, L215D, L215T, L215S, L215G, L215A, L215C, L215F, L215M, H221R and H221K.

The mutant may also comprise any combination of these mutations.

For example, the GTP cyclohydrolase I mutant may have at least 70% sequence identity with SEQ ID NO. 10, and comprise any one or more of the above mutations.

The GTP mutant may be the endogenous, native GTP cyclohydrolase which is mutated i.e. not an additional recombinant copy.

Additional Enzymes which Aid Tyrosine Hydroxylation Activity

In addition or as an alternative to the FolE mutation to increase co-factor production which in turn increases tyrosine hydroxylation, other enzymes in the pathway of FIG. 5b may be overexpressed or enzymes involved in the regeneration of the co-factor (such as 4a-hydroxytetrahydrobiopterin dehydratase encoded by the phhB gene).

For example, the microbial cell may over-express (compared to the wild-type under the same conditions) any nucleic acid encoding:
4a-hydroxytetrahydrobiopterin dehydratase (SEQ ID NO. 14): phhB (SEQ ID NO. 13); and/or
dihydroneopterin triphosphate 2′-epimerase (SEQ ID NO. 16): FoIX (SEQ ID NO. 15); and/or
dihydromonapterin reductase (SEQ ID NO. 12): FolM (SEQ ID NO. 11)

The nucleic acid may also be any encoding enzymes with these activities and having at least 70, 75, 80, 85, 90, 95 or 100% sequence identity with the above SEQ ID NO.s. The enzymes may additionally be truncated to the core secondary structure elements to provide function, for example by removing 1 to 20 (for example 1, 2, 3, 4, 5, 6, 7, 8, 9, 10, 11, 12, 13, 14, 15, 16, 17, 18, 19, 20) amino acids from the N and/or C termini of the constructs.

Upregulating expression may be via a recombinant nucleic acid, for example an additional copy of the gene on a plasmid or integrated into the genome, or alternatively via upregulating the endogenous sequence.

The microbial cell may have increased activity of FolE and/or FolM. Therefore, the microbial cell comprises a recombinant nucleic acid encoding a eukaryotic tyrosine hydroxylase (for example, a tyrosine hydrolase with at least 70% sequence identity to SEQ ID NO. 4) and upregulated FolE and/or FolM. This may be by additional recombinant FolE and/or FolM being added to the cell. The FolE enzyme may be mutated as described above.

Alternatively, the microbial cell comprises a recombinant nucleic acid encoding a eukaryotic tyrosine hydroxylase (for example, a tyrosine hydrolase with at least 70% sequence identity to SEQ ID NO. 4) and utilizes the endogenous FolE and FolM cofactors. The FolE enzyme may be mutated as described above.

Expression of the tyrosine hydroxylase (or example, a tyrosine hydrolase with at least 70% sequence identity to SEQ ID NO. 4) may be under a promoter comprising or consisting of consensus SEQ ID NO. 55. Expression of one or more of the co-factors (for example, FolE and/or FolM) may be under the control of a promoter comprising or consisting of SEQ ID NO. 56. The enzymes (and optionally the promoters described above) are preferably integrated into the genome of the cell.

Compound which Inhibits L-DOPA-Metabolizing Bacteria

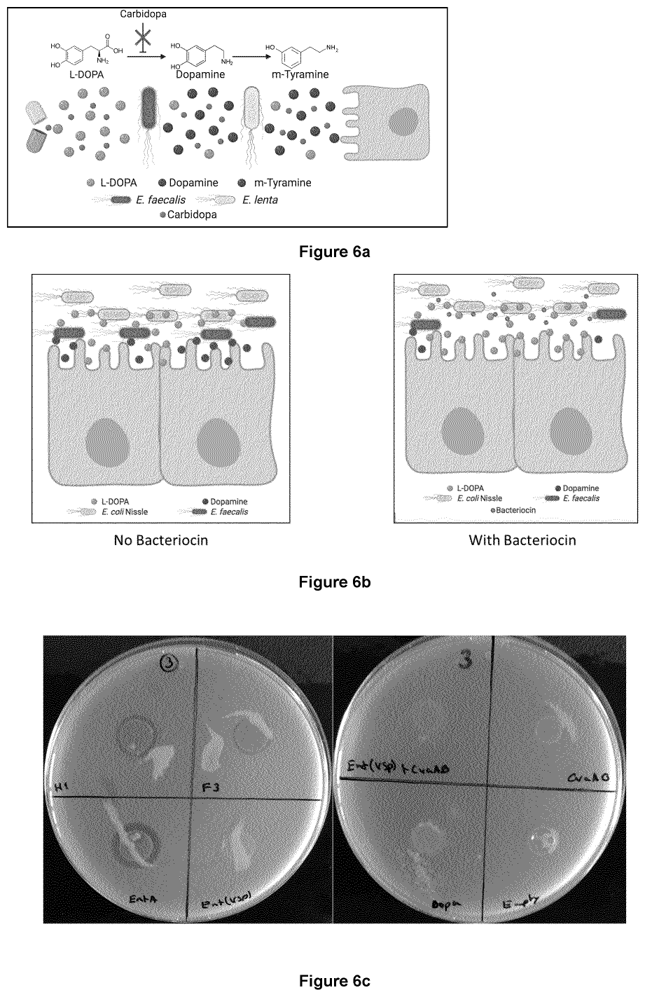

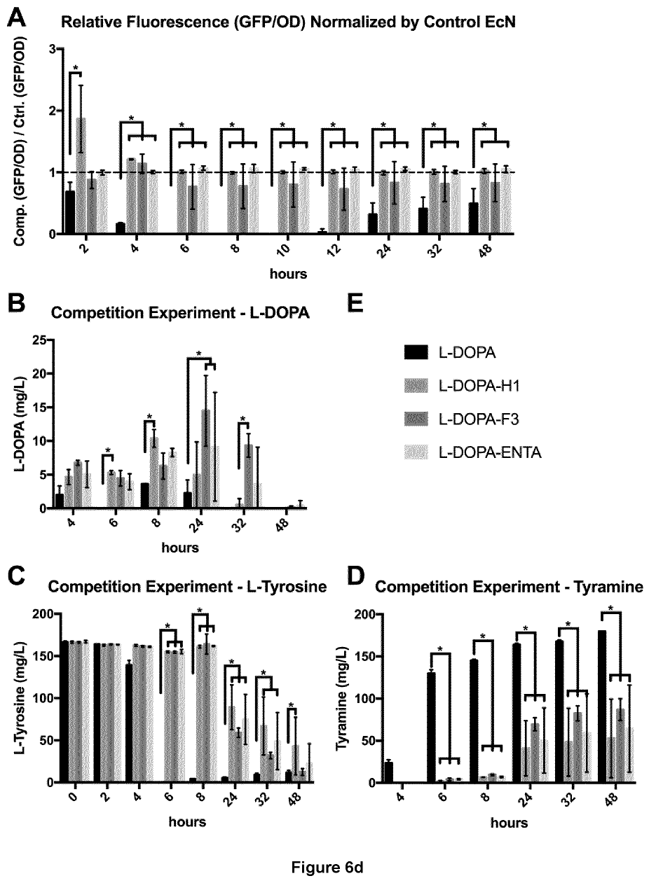

Bacteria such as E. faecalis metabolize L-DOPA in the gut (see FIG. 6b). To prevent this and to maintain L-DOPA concentrations, the microbial cell may be administered simultaneously, separately or sequentially with a compound which inhibits bacteria such as E. faecalis. Alternatively, the microbial cell may express the compound, or may be administered with a further microbial cell which expresses the compound. This administration may also be simultaneously, separately or sequentially.

The enzyme in E. faecalis responsible for metabolizing L-DOPA is TyrDC. Therefore, the compound may inhibit any bacteria which express TyrDC, for example, any bacteria comprising a nucleic acid encoding an enzyme with at least 70% sequence identity to SEQ ID No. 25.

Such a compound may be a bacteriocin. For example: Ubericin A, Hiracin, JM79 or Enterocin A (for example SEQ ID NO.s 29, 27 or 31). Alternatively the bacteriocin may be any of the below.

TABLE 1
Bacteriocins

| | | | | Uni |
|---|---|---|---|---|
| Accession | Name | Class | Producer organism | Prot |
| BAC006 | Subpeptin | Unclassified | Bacillus subtilis | P83879 |
| | JM4-B | | | |
| BAC028 | Variacin | Lantibiotic, Type A | Micrococcus varians | Q50848 |
| BAC065 | Curvacin-A | class IIA/YGNGV | Lactobacillus curvatus | P0A311 |
| BAC066 | Sakacin-A | class IIA/YGNGV | Lactobacillus sakei | P0A310 |
| | | | | P35619 |
| BAC078 | Sakacin-P | class IIA/YGNGV | Lactobacillus sakei | P35618 |
| | (Sakacin 674) | | | Q57121 |
| BAC088 | Enterocin A | Class IIa, IIc | Enterococcus faecium | Q47784 |
| | | (problematic) | (Streptococcus faecium) | |
| BAC092 | Lactacin-F | class IIB | Lactobacillus johnsonii | Q48509 |
| | (lafX) | | | |
| BAC098 | Subtilosin | Unclassified | Bacillus subtilis | Q7WY57 |
| BAC101 | Enterocin B | class IIc, non | Enterococcus faecium | O34017 |
| | | subgrouped | (Streptococcus faecium) | |
| | | bacteriocins | | |
| | | (problematic) | | |
| BAC105 | Lactacin-F | class IIB | Lactobacillus johnsonii | P24022 |
| | (lafA) | | | |
| BAC109 | Plantaricin W α | Lantibiotic (two- | Lactobacillus plantarum | Q9AF67 |
| | | peptide) | | |
| BAC113 | Cytolysin | Lantibiotic | Bacillus halodurans | Q9KFM6 |
| BAC114 | Plantaricin W β | Lantibiotic (two- | Lactobacillus plantarum | Q9AF68 |
| | | peptide) | | |
| BAC124 | Penocin A | class IIA/YGNGV | Pediococcus pentosaceus | Q03HX9 |
| | | | ATCC 25745 | |
| BAC133 | Enterolysin A | class III | Enterococcus faecalis | Q9F8B0 |
| | | | (Streptococcus faecalis) | |
| BAC141 | Aureocin A53 | Unclassified | Staphylococcus aureus | Q8GPI4 |
| BAC142 | Hiracin JM79 | Class II sec-dependent | Enterococcus hirae DCH5 | Q0Z8B6 |
| BAC143 | Enterocin AS-48 | Unclassified | Enterococcus faecalis | Q47765 |
| | (BACTERIOCIN | | (Streptococcus faecalis) | |
| | AS-48) | | | |
| BAC147 | Nisin U | Lantibiotic | Streptococcus uberis | Q2QBT0 |
| | | | ATCC 27958 | |
| BAC148 | Carnocyclin-A | Unclassified | Carnobacterium maltaromaticum | B2MVM5 |
| | | | (Carnobacterium piscicola) | |
| BAC149 | Enterocin 96 | Class II | Enterococcus faecalis | Q82YI9 |
| BAC150 | Ubericin A | Class IIa | Streptococcus uberis | A9Q0M7 |
| BAC156 | Bovicin HJ50 | Lantibiotic | Streptococcus bovis HJ50 | Q83ZN8 |
| BAC158 | Weissellicin 110 | Unclassified | Weissella cibaria 110 | No entry |
| | | | | found |
| BAC159 | Durancin TW-49M | Unclassified | Enterococcus durans QU 49 | B3IUC6 |
| BAC162 | Uberolysin | Unclassified | Streptococcus uberis | A5H1G9 |
| | | | strain 42 | |
| BAC167 | Bacteriocin T8 | class IIa | Enterococcus faecium T8 | Q27HG2 |
| BAC169 | Lacticin Q | Unclassified | Lactococcus lactis QU 5 | A4UVR2 |
| BAC178 | Leucocin Q | Class IId | Leuconostoc pseudomesenteroides | D7UPI8 |
| | | | QU 15 | |
| BAC179 | Leucocin N | Class IId | Leuconostoc pseudomesenteroides | D7UPI9 |
| | | | QU 15 | |
| BAC180 | Avicin A | class IIA/YGNGV | Enterococcus avium | D2DXK5 |
| BAC189 | Enterocin | Class IIb | Enterococcus faecium | D7UP03 |
| | Xalpha | | (Streptococcus faecium) | |
| BAC190 | Enterocin | Class IIb | Enterococcus faecium | D7UP04 |
| | Xbeta | | (Streptococcus faecium) | |
| BAC191 | Lactocyclicin Q | Unclassified | Lactococcus sp. QU 12 | B9ZZY0 |
| BAC192 | Garvicin ML | Unclassified | Lactococcus garvieae | D2KC49 |
| BAC200 | Weissellin A | Class IIa | Weissella | B3A0N4 |
| | | | paramesenteroides DX | |
| BAC201 | Thurincin H | Lantibiotic | Bacillus thuringiensis | B5U2V4 |
| | | | SF361 | |
| BAC203 | Enterocin | Unclassified | Enterococcus faecalis | Q8KMU4 |
| | EJ97 | | (Streptococcus faecalis) | |
| BAC209 | Leucocyclicin Q | Unclassified | Leuconostoc | G5ELQ0 |
| | | | mesenteroides TK41401 | |
| BAC210 | Epidermicin | Unclassified | Staphylococcus | H9BG66 |
| | NI01 | | epidermidis 224 | |
| BAC213 | Enterocin W | Class IIb | Enterococcus faecalis | H3JSS9 |
| | alfa | | (Streptococcus faecalis) | |
| BAC214 | Enterocin W | Class IIb | Enterococcus faecalis | H3JST0 |
| | beta | | (Streptococcus faecalis) | |
| BAC216 | Thuricin CD | two-peptide lantibiotic | Bacillus thuringiensis DPC | C2TQ80 |
| | alpha | | 6431 | |
| BAC217 | Thuricin CD | two-peptide lantibiotic | Bacillus thuringiensis DPC | C2TQ79 |
| | beta | | 6431 | |
| BAC219 | Garvicin A | IId | Lactococcus garvieae | H2B2W4 |
| BAC229 | Enterocin | Circular | Enterococcus faecalis | A0A0M3KKS4 |
| | NKR-5-3B | | NKR-5-3 | |
| | (Ent53B) | | | |
| BAC230 | Enterocin K1 | Leaderless | Enterococcus faecium | L2P7L3 |
| | | | EnGen0026 | |

The bacteriocin may also be any which has at least 70, 75, 80, 85, 90 or 95% sequence identity to any of the above bacteriocins. For example any of SEQ ID NO.s 27, 29 or 31.

Parkinson's Disease

Parkinson's disease causes impairment in both motor and non-motor functions. Current treatment is with L-DOPA in the form of tablet or inhalable powder.

Features Specific to Dopamine Production

Dopamine

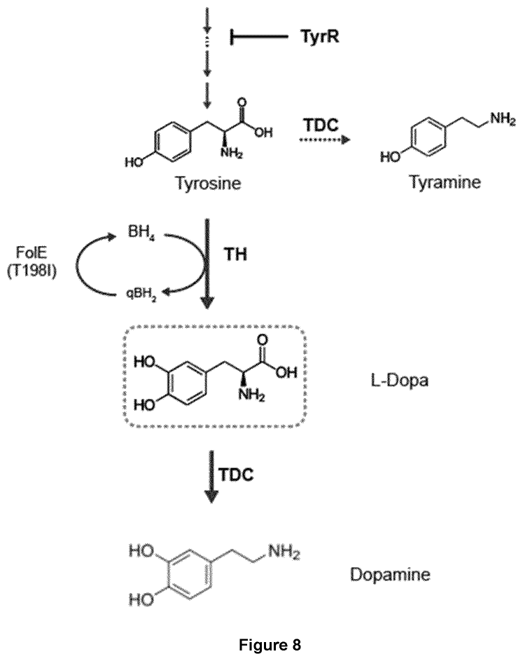

Dopamine is a hormone and a neurotransmitter that plays several important roles in the brain and body. It is an organic chemical of the catecholamine and phenethylamine families. It is an amine synthesized by removing a carboxyl group from a molecule of its precursor chemical L-DOPA. The structure of dopamine and the pathway from L-tyrosine is shown in FIG. 8.

Mutant Tyrosine Hydroxylase

The tyrosine hydroxylase may be a mutant, i.e. the enzyme differs from the full length wild type enzyme sequence.

The wild type full length rat enzyme comprises:

A regulatory domain (amino acids 1-154)
MPTPSAPSPQPKGFRRAVSEQDAKQAEAVTSPRFIGRRQSLIEDARKER
EAAAAAAAAAVASSEPGNPLEAVVFEERDGNAVLNLLFSLRGTKPSSLS
RAVKVFETFEAKIHHLETRPAQRPLAGSPHLEYFVRFEVPSGDLAALLS
SVRRVSD
A catalytic domain (amino acids 155-456)
DVRSAREDKVPWFPRKVSELDKCHHLVTKFDPDLDLDHPGFSDQVYRQR
RKLIAEIAFQYKHGEPIPHVEYTAEEIATWKEVYVTLKGLYATHACREH
LEGFQLLERYCGYREDSIPQLEDVSRFLKERTGFQLRPVAGLLSARDFL
ASLAFRVFQCTQYIRHASSPMHSPEPDCCHELLGHVPMLADRTFAQFSQ
DIGLASLGASDEEIEKLSTVYWFTVEFGLCKQNGELKAYGAGLLSSYGE
LLHSLSEEPEVRAFDPDTAAVQPYQDQTYQPVYFVSESFNDAKDKLRNY
ASRIQRPF
A tetramer domain (amino acids 457-498)
SVKFDPYTLAIDVLDSPHTIQRSLEGVQDELHTLAHALSAIS

The mutant may not comprise the regulatory domain. The entire regulatory domain may be deleted or only part of the regulatory domain may be deleted.

Truncation may be at any point in the regulatory domain to reduce the complexity of the protein for expression in a microbial cell and/or to decrease negative feedback by dopamine for the dopamine-producing microbial cell. The skilled person would be aware of suitable points to truncate the regulatory domain whilst maintaining the activity of the enzyme guided by the crystal structure (Goodwill, K., Sabatier, C., Marks, C. et al. Crystal structure of tyrosine hydroxylase at 2.3 Å and its implications for inherited neurodegenerative diseases. Nat Struct Mol Biol 4, 578-585 (1997).

The tyrosine hydroxylase may comprise the catalytic domain (and not the regulatory domain or tetramer domain); or the catalytic domain and the tetramer domain (and not the regulatory domain). These domains may comprise the above amino acids sequences or have at least 70, 75, 80, 85, 90, 95, 99 or 100% sequence identity with the above amino acid sequences, and optionally be further truncated to the core secondary structure elements to provide function, for example by removing 1-20 (for example 1, 2, 3, 4, 5, 6, 7, 8, 9, 10, 11, 12, 13, 14, 15, 16, 17, 18, 19 or 20) amino acids from the N and/or C termini of the constructs.

For example, the truncated enzyme may comprise the catalytic and tetramer domains, amino acids:

(amino acids 158-498 of SEQ ID NO. 2)
SAREDKVPWFPRKVSELDKCHHLVTKFDPDLDLDHPGFSDQVYRQRRKL
IAEIAFQYKHGEPIPHVEYTAEEIATWKEVYVTLKGLYATHACREHLEG
FQLLERYCGYREDSIPQLEDVSRFLKERTGFQLRPVAGLLSARDFLASL
AFRVFQCTQYIRHASSPMHSPEPDCCHELLGHVPMLADRTFAQFSQDIG
LASLGASDEEIEKLSTVYWFTVEFGLCKQNGELKAYGAGLLSSYGELLH
SLSEEPEVRAFDPDTAAVQPYQDQTYQPVYFVSESFNDAKDKLRNYASR
IQRPFSVKFDPYTLAIDVLDSPHTIQRSLEGVQDELHTLAHALSAIS.

Optionally the truncated enzyme may be SEQ ID NO. 4.

Alternatively, the truncated enzyme may comprise the catalytic domain only:

SAREDKVPWFPRKVSELDKCHHLVTKFDPDLDLDHPGFSDQVYRQRRKLIAEIAFQYKHGE PIPHVEYTAEEIATWKEVYVTLKGLYATHACREHLEGFQLLERYCGYREDSIPQLEDVSRFL KERTGFQLRPVAGLLSARDFLASLAFRVFQCTQYIRHASSPMHSPEPDCCHELLGHVPMLA DRTFAQFSQDIGLASLGASDEEIEKLSTVYWFTVEFGLCKQNGELKAYGAGLLSSYGELLHS LSEEPEVRAFDPDTAAVQPYQDQTYQPVYFVSESFNDAKDKLRNYASRIQRPF (amino acids 158-456 of SEQ ID NO. 2). Optionally the truncated enzyme may be amino acids 1-301 of SEQ ID NO. 4.

The tyrosine hydroxylase may be any sequence having at least 70, 75, 80, 85, 90 or 95% sequence identity to the above truncated forms. The enzyme may additionally be truncated to the core secondary structure elements to provide function, for example by removing 1 to 20 (for example 1, 2, 3, 4, 5, 6, 7, 8, 9, 10, 11, 12, 13, 14, 15, 16, 17, 18, 19, 20) amino acids from the N and/or C termini of the construct.

The truncated forms described above may be used for L-DOPA as well as dopamine production.

The following mutants are particularly adapted for dopamine production.

The tyrosine hydroxylase may alternatively or additionally be mutated to increase flux through the pathway and/or to prevent dopamine inhibition of tyrosine hydroxylase.

The tyrosine hydroxylase may not comprise an active regulatory domain meaning the regulatory domain is mutated to prevent feedback inhibition by dopamine.

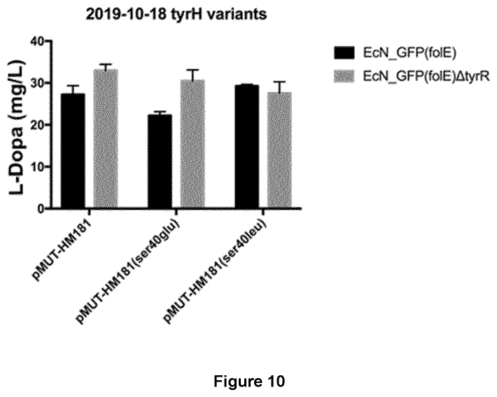

The tyrosine hydroxylase may alternatively or additionally comprise a mutation in the catalytic domain which increases dopamine production, for example by 3-fold compared to the wild type. The mutation may be in amino acids 177-198 of SEQ ID NO. 2. These amino acids form a loop as shown by the crystal structure of the enzyme. The inventors have surprisingly found that mutating an amino acid in this loop increases dopamine production. The amino acid mutated in this loop may be at position 196. The mutant may be Ser 196Glu or Ser196Leu. These are shown below in the rat full length enzyme, and truncated enzyme. The mutation in the truncated form corresponds to position 41, optionally to Glu/Leu (Ser 40 without the start codon, and as referred to in FIG. 10).

Full Length Mutant (Loop 177-198 is Underlined; Mutation 196 is in Brackets)

MPTPSAPSPQPKGFRRAVSEQDAKQAEAVTSPRFIGRRQSLIEDARKER
EAAAAAAAAAVASSEPGNPLEAVVFEERDGNAVLNLLFSLRGTKPSSLS
RAVKVFETFEAKIHHLETRPAQRPLAGSPHLEYFVRFEVPSGDLAALLS
SVRRVSDDVRSAREDKVPWFPRKVSELDKCHHLVTKFDPDLDLDHPGF
[E/L]DQVYRQRRKLIAEIAFQYKHGEPIPHVEYTAEEIATWKEVYVTL
KGLYATHACREHLEGFQLLERYCGYREDSIPQLEDVSRFLKERTGFQLR
PVAGLLSARDFLASLAFRVFQCTQYIRHASSPMHSPEPDCCHELLGHVP
MLADRTFAQFSQDIGLASLGASDEEIEKLSTVYWFTVEFGLCKQNGELK
AYGAGLLSSYGELLHSLSEEPEVRAFDPDTAAVQPYQDQTYQPVYFVSE
SFNDAKDKLRNYASRIQRPFSVKFDPYTLAIDVLDSPHTIQRSLEGVQD
ELHTLAHALSAIS

Truncated Mutant without the Regulatory Domain (Loop 22-43 is Underlined; Mutation 41 is in Brackets

(SEQ ID NO.s 6 and 8)
MKSAREDKVPWFPRKVSELDKCHHLVTKFDPDLDLDHPGF[E/L]DQVY
RQRRKLIAEIAFQYKHGEPIPHVEYTAEEIATWKEVYVTLKGLYATHAC
REHLEGFQLLERYCGYREDSIPQLEDVSRFLKERTGFQLRPVAGLLSAR
DFLASLAFRVFQCTQYIRHASSPMHSPEPDCCHELLGHVPMLADRTFAQ
FSQDIGLASLGASDEEIEKLSTVYWFTVEFGLCKQNGELKAYGAGLLSS
YGELLHSLSEEPEVRAFDPDTAAVQPYQDQTYQPVYFVSESFNDAKDKL
RNYASRIQRPFSVKFDPYTLAIDVLDSPHTIQRSLEGVQDELHTLAHAL
SAIS

This mutation at position 196 in the full length or 41 in the truncated form may also be applied to any of the truncated mutants above, for example the truncated form comprising only the catalytic domain.

Therefore, the tyrosine hydroxylase may comprise any of the truncated forms above and additionally comprise a mutation in the loop: CHHLVTKFDPDLDLDHPGFSDQ, optionally at the underlined serine position.

For example, the mutant may be SEQ ID NO. 6 or 8, or a mutant with at least 70, 75, 80, 85, 90 or 95% sequence identity to SEQ ID NO. 6 or 8.

The tyrosine hydroxylase may have at least 70, 75, 80, 85, 90, 95 or 100% sequence identity with any of the above mutant forms. Additionally, the mutant may be further truncated to the core secondary structure elements to provide function, for example by removing 1 to 20 amino acids (for example, 1, 2, 3, 4, 5, 6, 7, 8, 9, 10, 11, 12, 13, 14, 15, 16, 17, 18, 19 or 20) from the N and/or C termini of the constructs.

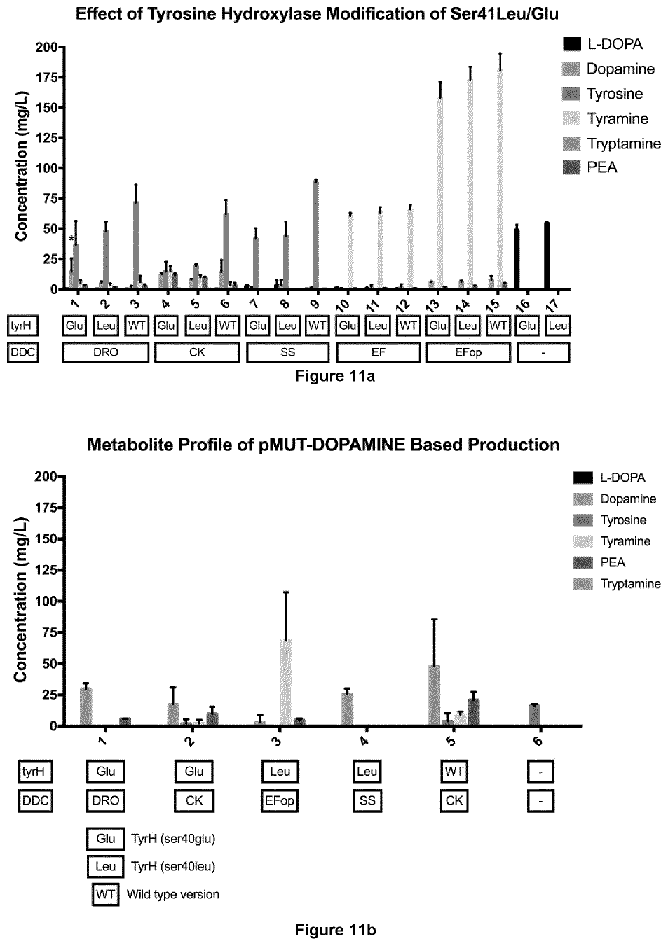

The inventors have surprisingly found that the above mutants (with the mutation at position 196 in the full length sequence and position 41 in the truncated sequence without the regulatory domain) produced less L-DOPA, for example 5, 10, 15 or 20% less L-DOPA compared to the wild-type, but at least 1.5 fold, 2 fold, 2.5 fold or 3 fold higher dopamine. This is set out in the table below and FIG. 11 (in FIG. 11a, TH(ser196leu)+SS decarboxylase produces 3.16 mg/L in comparison to 0.93 mg/L of the WT TH+SS decarboxylase).

TABLE 2
L-DOPA production by mutants (TyrR
refers to Tyrosine Repressor)

| tyrH variant in Nissle with GFP integrated, | |
|---|---|
| folE(T198I) and tyrR KO | L-DOPA (mg/L) |
| WT TH (truncated) | 32.99 |
| WT TH ser40glu (truncated) | 30.46 |
| WT TH ser40leu (truncated) | 27.56 |

Also see FIG. 10 which shows L-DOPA production from the Ser40 mutation (Ser41 including the start codon).

L-DOPA Decarboxylase Activity

To produce dopamine from L-DOPA, the L-DOPA is decarboxylated.

The L-DOPA decarboxylase used may be any of the following:
SS (Sus scrofa) DDC: EC:4.1.1.28. SEQ ID NO. 18;
CK (Koribacter versatilis) DDC: EC:4.1.1.28; EC:4.1.1.105. SEQ ID NO. 20;
DRO (Draconibacterium orientale) DDC: EC:4.1.1.28; EC:4.1.1.105. SEQ ID NO. 22;
EF (Enterococcus Faecalis) DDC: EC:4.1.1.25. SEQ ID NO. 25.

The decarboxylase may also be any with at least 70, 75, 80, 85, 90, 95, 97 or 99% sequence identity with the above enzymes. The enzyme may additionally be truncated to the core secondary structure elements to provide function, for example by removing 1 to 20 (for example 1, 2, 3, 4, 5, 6, 7, 8, 9, 10, 11, 12, 13, 14, 15, 16, 17, 18, 19, 20) amino acids from the N and/or C termini of the construct.

Dopamine-Related Disorders

Peripheric dopamine can affect browning of adipocytes, energy expenditure, levels of glucose in blood and contribute to insulin signaling. Therefore, the microbial cell expressing dopamine may help treat diabetes, obesity and/or other metabolic diseases.

Furthermore, Dopamine modulates the immune system. Therefore, the microbial cell expressing dopamine could be to regulate the immune response in the gut. For example, the microbial cell could be used to treat Irritable bowel disease, ulcerative colitis, Chrohn's disease, Intestinal cancers.

The microbial cell may also be used to treat other immune-mediated inflammatory diseases. For example, the microbial cell may be used to treat ankylosing spondylitis, psoriasis, psoriatic arthritis, Behcet's disease, arthritis and allergy.

Dopamine can also regulate blood pressure. Therefore, the microbial cell may be used as a blood pressure modulators. For example, the microbial cell may be used to treat high or low blood pressure.

As L-DOPA can be converted into dopamine peripherally, the L-DOPA producing microbial cells can also be used to deliver dopamine and hence treat any of the dopamine-related disorders above.

Throughout the specification, unless the context demands otherwise, the terms ‘comprise’ or ‘include’, or variations such as ‘comprises’ or ‘comprising’, ‘includes’ or ‘including’ will be understood to imply the method or kit includes a stated integer or group of integers, but not the exclusion of any other integer or group of integers.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness. Reference to cited material or information contained in the text should not be understood as a concession that the material or information was part of the common general knowledge or was known in any country.

TABLE 3
Sequence listings

| | DNA | Amino acid |
|---|---|---|
| Name | SEQ ID NO. | SEQ ID NO. |
| Rat Tyrosine Hydroxylase | SEQ ID NO. 1 | SEQ ID NO. 2 |
| Truncated Tyrosine | SEQ ID NO. 3 | SEQ ID NO. 4 |
| Hydroxylase | | |
| Truncated and mutated | | |
| Tyrosine Hydroxylase | | |
| Ser 41 to Glu | 1) SEQ ID NO. 5 | 1) SEQ ID NO. 6 |
| Ser 41 to Leu | 2) SEQ ID NO. 7 | 2) SEQ ID NO. 8 |
| GTP cyclohydrolase (FolE) | SEQ ID NO. 9 | SEQ ID NO. 10 |
| FolE codon optimized | SEQ ID NO. 51 | SEQ ID NO. 52 |
| folM | SEQ ID NO. 11 | SEQ ID NO. 12 |
| FolM codon optimized | SEQ ID NO. 53 | SEQ ID NO. 54 |
| phhB | SEQ ID NO. 13 | SEQ ID NO. 14 |
| folX | SEQ ID NO. 15 | SEQ ID NO. 16 |
| Promoter: | | |
| 1) trc promoter | 1) SEQ ID NO. 34 | |
| 2) trc promoter with lac | 2) SEQ ID NO. 35 | |
| operator | | |
| 3) trc promoter without lac | 3) SEQ ID NO. 36 | |
| operator (without lacl | | |
| binding site) | | |
| 4) BBa_J23100 | 4) SEQ ID NO. 37 | |
| 5) J23102 | 5) SEQ ID NO. 38 | |
| 6) MSKL7 | 6) SEQ ID NO. 32 | |
| 7) MSKL8 | 7) SEQ ID NO. 33 | |
| 8) J23101 | 8) SEQ ID NO. 39 | |
| 9) J23105 | 9) SEQ ID NO. 40 | |
| 10) J23106 | 10) SEQ ID NO. 41 | |
| 11) J23107 | 11) SEQ ID NO. 42 | |
| 12) J23108 | 12) SEQ ID NO. 43 | |
| 13) J23110 | 13) SEQ ID NO. 44 | |
| 14) J23111 | 14) SEQ ID NO. 45 | |
| 15) J23114 | 15) SEQ ID NO. 46 | |
| 16) J23115 | 16) SEQ ID NO. 47 | |
| 17) J23116 | 17) SEQ ID NO. 48 | |
| 18) J23117 | 18) SEQ ID NO. 49 | |
| 19) J23118 | 19) SEQ ID NO. 50 | |
| 20) MSKL consensus | 20) SEQ ID NO. 55 | |
| 21) Anderson consensus | 21) SEQ ID NO. 56 | |
| Decarboxylase: | | |
| 1) SS | 1) SEQ ID NO. 17 | 1) SEQ ID NO. 18 |
| 2) CK | 2) SEQ ID NO. 19 | 2) SEQ ID NO. 20 |
| 3) DRO | 3) SEQ ID NO. 21 | 3) SEQ ID NO. 22 |
| 4) EF TyrDC | 4) SEQ ID NO. 23 | 4) SEQ ID NO. 25 |
| 5) EF TyrDC optimized | 5) SEQ ID NO. 24 | 5) SEQ ID NO. 25 |
| Bacteriocins: | | |
| 1) Hiracin JM79 | 1) SEQ ID NO. 26 | 1) SEQ ID NO. 27 |
| 2) Ubericin A | 2) SEQ ID NO. 28 | 2) SEQ ID NO. 29 |
| 3) Enterocin A | 3) SEQ ID NO. 30 | 3) SEQ ID NO. 31 |

Aspects of the present invention will now be illustrated by way of example only and with reference to the following experimentation.

### EXAMPLES

Strains and Cultivation Conditions

For general lab procedures strains were grown using LB media at 37° C., unless otherwise stated. Strains generated were stored at −80° C., glycerol stocks (glycerol 25%). Proper antibiotics were used accordingly to the resistance markers of the different strains.

L-DOPA Production Cultures

L-DOPA production cultures were carried out in 96 deep well plates and 350 μl media. Biological triplicates of each strain were used to inoculate precultures in M9 media with 0.4% glucose with or without 0.2% CAS amino acids and L-Tyrosine. Precultures were grown for 24 hours at 37° C. in a shaking incubator at 250 RPM. Production cultures were carried by inoculating the preculture with 1:100 ratio of the total volume and incubated at 37° C. in a shaking incubator at 250 RPM for 22 hours. After 22 hours the cultures were centrifuged at 4700 RPM and the supernatant was collected and frozen until further analysis.

Plasmids

Plasmid Construction and Purification

L-DOPA and dopamine producing plasmids were constructed using USER cloning. pMUT plasmid, truncated tyrosine hydroxylase, decarboxylases and other genes were amplified using Phusion U polymerase and uracil containing primers. These fragments were later purified using Thermofisher PCR purification kit and were subsequently cloned together using the USER enzyme. Top10 chemically competent cells were transformed by heat-shock with 5 μl of USER reaction and plated in LB plates supplemented with kanamycin. Plates were incubated at 37° C. overnight. Correct constructs were screen by colony PCR and confirmed by sequencing. Correct colonies were incubated in 2×YT supplemented with kanamycin at 37° C. overnight. Plasmids were later extracted from the cultures using MACHEREY-NAGEL plasmid purification kit.

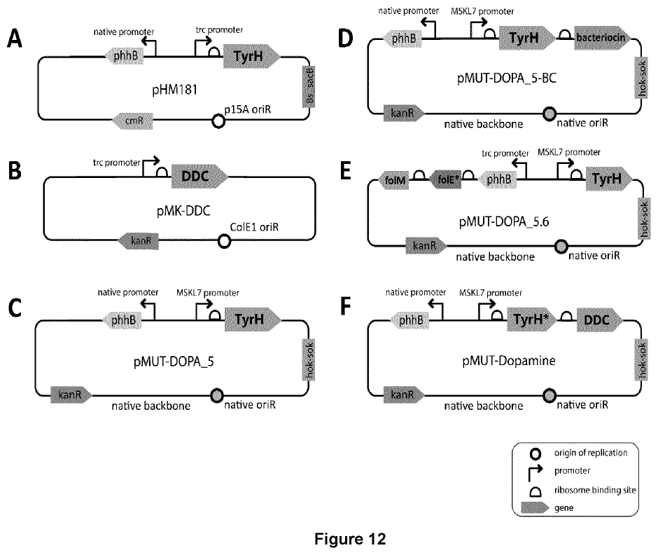

The plasmids are shown in FIG. 12.

The truncated TH, phhB and all DDCs except for EF have been codon optimized. folE, folM and folX are native sequences from E. coli.

TABLE 4
Plasmids for L-DOPA production, referred to in the examples and figures

| | Strain name (in figures and | | |
|---|---|---|---|
| FIG. | examples) | Genotype | Plasmid contains |
| 3-A | Nissle | Nissle with GFP | No plasmid |
| | 100 mg/L tyrosine was | integrated | |
| | supplemented | | |
| 3-A | Nissle + pDOPA | Nissle with GFP | pHM 181 (plasmid |
| | 100 mg/L tyrosine was | integrated | transferred from I- |
| | supplemented | | loop) FIG. 12.A |
| 3-A | Nissle(folE) + pDOPA | Nissle with GFP | pHM181 (plasmid |
| | 100 mg/L tyrosine was | integrated and | transferred from I- |
| | supplemented | folE(T198I) | loop) FIG. 12.A |
| 3-B | Nissle(folE) + pDOPA | Nissle with GFP | pHM181 (plasmid |
| | Different amounts L-Tyrosine | integrated and | transferred from I- |
| | were supplemented (0, 20, | folE(T198I) | loop) FIG. 12.A |
| | 50, 100 mg/L) | | |
| 3-C | Black columns: Nissle(folE) | Black columns: | All plasmid here are |
| | Grey columns: | Nissle with GFP | pMUT versions |
| | Nissle(folE),tyrR-KO | integrated and | (Nissle native |
| | | folE(T198I) | plasmid) |
| | | Grey columns: | Only plasmid that is |
| | | Nissle with GFP | not explained is |
| | | integrated, | pDOPA_1, which is |
| | | folE(T198I) and tyrR | PMUT-HM181, all |
| | | KO | others are variations |
| | | | (different promoters) |
| | | | of this one. |
| | | | pDOPA_1(IPTG) |
| | | | means that IPTG was |
| | | | supplemented to this |
| | | | culture only). |
| 4 | EcN_GFP(folEmut) | Nissle with GFP | No phhB = pHM181 |
| | | integrated and | with no phhB gene |
| | | folE(T198I) | PhhB = pHM181 (it |
| | | | should contain pHHB) |
| 5-A | EcN_GFP(folE)ΔtyrR | Nissle with GFP | pMUT-HM181_5 = |
| | | integrated, | pMUT-DOPA_5 |
| | | folE(T198I) and tyrR | FIG. 12.C |
| | | KO | variations: |
| | | | .4 = changed |
| | | | promoter of pHHB for |
| | | | the trc promoter |
| | | | .5 = added |
| | | | folE(T198I) gene from |
| | | | E. coli Nissle |
| | | | .6 = added folE(T198) |
| | | | and folM from Nissle. |
| | | | FIG. 12.E |
| | | | M9(GLU + CasA) = M9 |
| | | | medium + 0.4% |
| | | | Glucose and 0.2% |
| | | | Cas Amino acids |
| | | | M9(GLU) = M9 |
| | | | medium + 0.4% |
| | | | Glucose |
| 6C | H1 | EcN_GFP(folE)ΔtyrR | H1 = pMUT-DOPA_5-H1 |
| | | | FIG. 12.D |
| 6C | F3 | EcN_GFP(folE)ΔtyrR | F3 = pMUT-DOPA_5-F3 |
| | | | FIG. 12.D |
| 6C | EntA | EcN_GFP(folE)ΔtyrR | EntA = pMUT-DOPA_5- |
| | | | EntA FIG. 12.D |
| 6C | DOPA | EcN_GFP(folE)ΔtyrR | Dopa = pMUT-DOPA_5 |
| | | | FIG. 12.C |
| 6C | Empty | EcN_GFP(folE)ΔtyrR | Empty = pMUT-Empty |
| 7 | EcN_CTRL/EcN_WT | EcN_GFP(folE)ΔtyrR | pMUT-Empty |
| 7 | EcN_DOPA/EcN_L-DOPA | EcN_GFP(folE)ΔtyrR | pMUT-DOPA_5.6 |

TABLE 5
Plasmids for Dopamine production, referred to in the examples and figures

| | Strain name (in figures and | | |
|---|---|---|---|
| FIG. | examples) | Genotype | Plasmid contains |
| 9 | EcN_GFP(folEmut) | Nissle with GFP | All strains here have |
| | 100 mg/L tyrosine was | integrated and | the pHM181 plasmid |
| | supplemented | folE(T198I) | (FIG. 12.A) + the |
| | | | pMK plasmid with |
| | | | different |
| | | | Decarboxylases (2 |
| | | | plasmids in total) |
| | | | (FIG. 12.B) |
| 10 | Black columns: Nissle(folE) | Black columns: | pMUT-HM181 or |
| | Grey columns: | Nissle with GFP | pMUT-DOPA_1 with |
| | Nissle(folE), tyrR-KO | integrated and | different variations of |
| | | folE(T198I) | TH. |
| | | Grey columns: | |
| | | Nissle with GFP | |
| | | integrated, | |
| | | folE(T198I) and tyrR | |
| | | KO | |
| 11a | EcN_GFP(folEmut) | Nissle with GFP | All strains here have |
| | 100 mg/L tyrosine was | integrated and | the pHM181 plasmid |
| | supplemented | folE(T1981) | with the 40GLU and |
| | | | 40LEU variation |
| | | | FIG. 12.A + the |
| | | | pMK plasmid with |
| | | | different |
| | | | Decarboxylases (2 |
| | | | plasmids in total) |
| | | | FIG. 12.B |
| 11b | EcN_GFP(folE)ΔtyrR | Nissle with G*Fp | Plasmid: pMUT |
| | No tyrosine was | integrated, | based production of |
| | supplemented | folE(T198I) and tyrR | dopamine (1 plasmid |
| | | KO | only). The plasmid |
| | | | has the TH either in |
| | | | WT, 40GLU or 40LEU |
| | | | form and |
| | | | decarboxylase |
| | | | downstream. Figure |
| | | | 12.F |
| | | | Last strain (- -) has |
| | | | the pMUT empty |
| | | | plasmid no TH not |
| | | | decarboxylase |

Transformation of E. coli Nissle

1 colony of E. coli Nissle was grown overnight at 37° C. in a shaking incubator. Next day, 1:100 dilution was inoculated in 10 ml of 2×YT for 3-4 hours. At OD₆₀₀=0.4-0.5 cells were harvested, washing 3 times with cold 10% glycerol in MQ water, and were later electroporated using Bio-RAD MicroPulser electroporator and 0.1 mm electroporation cuvettes. Transformants cells were recovered in 1 ml of SOC media at 37° C. for 1 hour before plating them in LB plates supplemented with kanamycin and incubated at 37° C. overnight.

### Examples 1-4 Relate to L-DOPA Production

### Example 1: Bacteria can Express Large Quantities of L-DOPA Via a Eukaryotic Tyrosine Hydroxylase

E. coli Nissle strains were inoculated in biological triplicates and grown for 24 hours in M9 media with 0.4% glucose (Preculture). Production culture was inoculated in 1:100 ratio from the preculture and grown for 22 hours in M9 media with 0.4% glucose and supplemented with 100 mg/L of L-Tyrosine. Production cultures were centrifuged at 4500 RPMs and supernatant was collected for H PLC analysis.

HPLC analysis was carried out as follows:

Quantitative analysis of L-DOPA, L-Tyrosine and dopamine in cell-free supernatant was performed by High-Performance Liquid Chromatography (HPLC) on an UltiMate 3000 UHPLC system (ThermoScientific). The system consisted of an LPG-3400RS quaternary pump and a WPS-3000RS autosampler with a TCC-3000 column oven and a DAD-3000 diode array detector. Samples were run at a pressure of 600 bar through a CORTECS column (1.6 μm, 2.1×150 mm) at 30° C. with an injection volume of 1 μl and a flowrate of 0.350 ml/min in 10 mM ammonium formate as mobile phase.

Constructing Promoter Variants

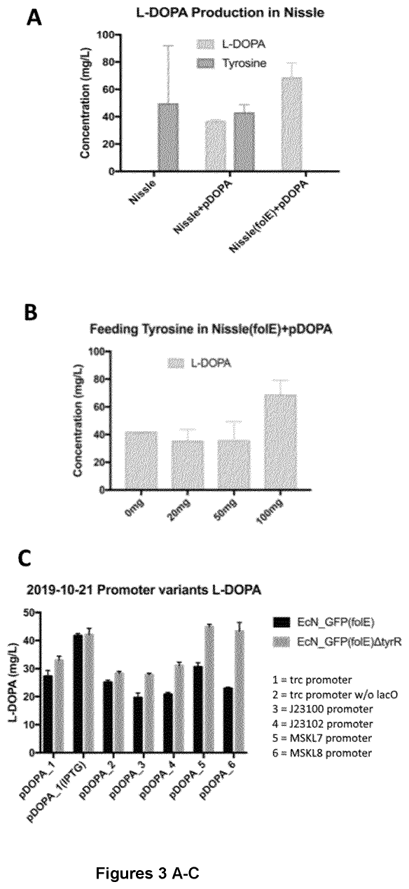

pHM181 (=pDOPA_1) was used as the starting point from which all other variants tested in FIG. 3C were created. In pHM181, the truncated TyrH gene is under control of the IPTG-inducible trc promoter, which contains a lac operator for repression by the Lacl repressor. Plasmids pDOPA_2 to pDOPA_6 were constructed by modifying or replacing the trc promoter on the plasmid, employing USER cloning. In pDOPA_2, part of the promoter (the lac operator) was removed. In pDOPA_3-6, the trc promoter was replaced with the promoters shown in Table 6 below.

TABLE 6
Promoters tested

| Promoter | | |
|---|---|---|
| name | Sequence | Features |
| trc | ttgacaattaatcatccggctcgtataatg | IPTG-inducible, catabolite- |
| promoter | | repressed promoter |
| trc | ttgacaattaatcatccggctcgtataatgtgtggaattg | With lacI binding site |
| promoter | tgagcggataacaatttcacacaggagtaaaa | |
| with lac | | |
| operator | | |
| trc | ttgacaattaatcatccggctcgtataatgtgtggaattt | lacI binding site removed |
| promoter | cacacaggagtaaaa | |
| without lac | | |
| operator | | |
| BBa_J23100 | ttgacggctagctcagtcctaggtacagtgctagc | constitutive promoter. Strongest |
| | | promoter from BioBricks library |
| Ba_J23102 | ttgacagctagctcagtcctaggtactgtgctagc | constitutive promoter. Second- |
| | | strongest promoter from |
| | | BioBricks library |
| MSKL7 | tgcttgactcgtcgttcctcctacgtgtataattgg | constitutive promoter, optimized |
| | | for in vivo application (ref: Novel |
| | | High-Throughput Methods for |
| | | Rapid Development of Cell |
| | | Factories. PhD Thesis, MS |
| | | Klausen, 2019). Second- |
| | | strongest from library |
| MSKL8 | tgcttgactcgtcgttatcctacgtgtataattggc | constitutive promoter, optimized |
| | | for in vivo application (ref: Novel |
| | | High-Throughput Methods for |
| | | Rapid Development of Cell |
| | | Factories. PhD Thesis, MS |
| | | Klausen, 2019). Strongest from |
| | | library |

Production of L-DOPA

Biological triplicates of E. coli Nissle strains harboring the different promoter constructs were grown, using 96 deep-well plates, in 350 μl of M9 minimal media with 0.4% glucose for 24 hours in a shaking incubator at 37° C. and 250 RPM. Production culture was inoculated with 1:100 inoculum from the preculture in fresh M9 minimal media with 0.4% glucose. The plate was incubated for 22 hours at 37° C. and 250 RPM. The production culture was then centrifuged at 4500 RPM and supernatants were transferred into a 96 well microtiter plate and stored at −20° C. until HPLC analysis.

Results Summary

The results are shown in FIG. 3.

FIGS. 3a and b show production of L-DOPA (measured by LC-MS). FIG. 3c shows the strains produce at least 30 mg/I in minimal media measured by H PLC).

The FolE mutant shows increased production as seen in FIG. 3a. Additionally, we show the strain is able to produce L-DOPA from glucose with no supplement of tyrosine (FIG. 3b), which is an important requirement for being functional in vivo.

Promoter MSKL7 and MSKL8 with the tyrR KO produce the most L-DOPA. Promoter 7 was chosen for further experiments as it showed an increase of L-DOPA production in both genotypes with and without tyrR KO compared to promoter 8.

### Example 2: Optimisation of Co-Factor Production

The same cultivation method as previously described was used to test the effect of changes to the co-factor production on the amount of L-DOPA produced.

The following differences in plasmids were tested:

pMUT-DOPA_5 is shown in FIG. 12

Variations made to this plasmid were as follows:

5.4=changed promoter of phhB for the trc promoter

5.5=added folE(T198I) gene from E. coli Nissle

5.6=added folE(T198I) and folM from Nissle (also shown in FIG. 12).

Additionally, a further experiment was carried out to probe the effect of over-expression of phhB.

Results Summary

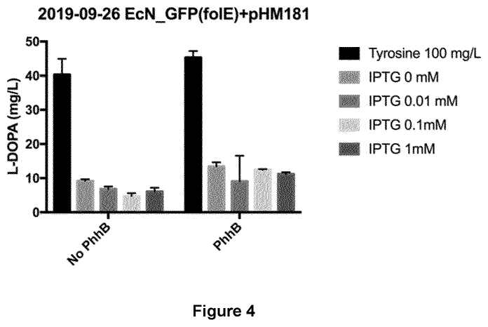

The results are shown in FIGS. 4 and 5a.

In addition to folE, the addition of folM also increase L-DOPA production.

With regards to phhB, it was found that in a background strain containing the genomic folE(T198I) mutation, co-expression of phhB increases L-DOPA production in a small but statistically significant manner (FIG. 4). Therefore, the phhB gene was incorporated into the final DOPA and Dopamine production constructs.

### Example 3: Bacteriocins Inhibit E. faecalis in the Region of the L-DOPA Producing Bacteria

Bacteriocin Assay (FIG. 6C)

Three colonies of each E. coli Nissle strain were inoculated in 5 ml of BHI broth and grown overnight at 37° C. in a shaking incubator. The following day, 1 ml of the culture was washed with PBS buffer once and 10 μl were used to spot the strains on top of a BHI agar plate. After drying, top BHI agar was mixed with 500 μl of previously grown E. faecalis, and was placed on top of the BHI agar containing the dried spots of the E. coli strains. Plates were dried for 10 minutes and then incubated at 37° C. overnight.

Competition Experiments (FIG. 6D)

E. faecalis and L-DOPA EcN strains expressing different bacteriocins were grown overnight in Brain Heart Infusion (BHI) broth (NutriSelect™), without supplementation of antibiotics. The next day, EcN cultures were washed once and resuspended in PBS. Cultures were diluted accordingly to have a concentration of 10^{\hat{ }7} and 10^{\hat{ }6} CFU/ml of EcN and E. faecalis respectively in 10 ml of BHI. Throughout the experiment 200 μl were taken periodically for CFU plating and 1 ml for future HPLC quantification. Samples for HPLC quantification were centrifuged at 10 000 g for 3 min, supernatant was transferred into a 96-well plate. Before HPLC quantification the supernatants were filtered using a 96-well filter plate (AcroPrep™) Culture dynamics were followed by transferring 200 μl of the competition culture into a 96-well microtiter plate and running a kinetic experiment measuring OD and GFP in a fluorescent microtiter plate reader (Synergy H1). Competition experiment was performed for 48 hours.

Results Summary

The results are shown in FIGS. 6c and 6d.

FIG. 6C shows halos of inhibition in Brain Heart Infusion (BHI) media from E. faecalis surrounding L-DOPA producing E. coli Nissle spots and co-expressing bacteriocins (Hiracin JM79, ubericin A and Enterocin A).

FIG. 6D shows the following:

6D-A: L-DOPA producing EcN strains, which co-express bacteriocins outcompete E. faecalis compared to an L-DOPA producing strain, which does not produce bacteriocins.

6D-B these strains also are able to maintain higher levels of L-DOPA in the supernatant and for longer time than the EcN that does not produce bacteriocins.

6D-C,D These strains inhibit the metabolism of tyrosine into tyramine by E. faecalis. The enzyme tyrDC, responsible for this is also the one that turns L-DOPA into dopamine and contributes to the degradation of L-DOPA and a poor therapeutic response in PD patients.

These results show the strain can not only express L-DOPA but also inhibit E. faecalis in the vicinity of the L-DOPA producing strain meaning higher levels of L-DOPA can be maintained instead of being metabolised to dopamine.

### Example 4: In Vivo Results

Oral Gavage of Engineered E. coli Nissle:

Female mice (NMRI, supplied by Taconic Biosciences, 6 weeks of age) were group-housed on a 12-h light:dark cycle at constant temperature with ad libitum access to food and water in a Specific Pathogen Free (SPF) facility. Upon delivery, mice were given 5 days to adjust to new location. Cohort size was 8 animals, and 4 different cohorts were tested, see below. All animals received Streptomycin (5 g/L) in the drinking water to ensure colonization, 3 days before being gavaged and throughout the experiment. A single oral gavage of 10⁸ cells was administered of either L-DOPA-producing (called ‘EcN_DOPA’) or a control E. coli Nissle (‘EcN_CTRL’) strain without expression of the tyrosine hydroxylase gene. Samples were taken for the following 7 days, after which animals were euthanized and final blood samples and gut content samples were collected. 2 of the 4 cohorts were also treated with the TDC inhibitor Carbidopa via intraperitoneal injection (10 mg/kg body weight) every 24 h. Fresh fecal samples were collected daily for 7 days to quantify colonization and metabolite levels. Plasma samples were taken on day 2 (submandibular sampling) and day 7 (vena cava) after gavage, and urine samples were taken on day 3 and 6.

In vivo sample analysis: Plasma, tissue samples, gut content and fecal samples were analyzed for DOPA-derived and related serotonin metabolites using LC-MS. For plasma: blood samples were collected using BD microtainer tubes with Li-Heparin coating, and plasma was prepared according to the manufacturer's instructions and frozen at −80 C. Urine samples were collected within 30 minutes of urination and immediately frozen at −80 C. Both sample types were then thawed, mixed with an internal standard buffer (IS buffer) containing 0.9% NaCl, 0.2% Ascorbic acid and 20 mg/L C¹³, N¹⁵-labelled Tryptophan, and then methanol-precipitated. After drying samples using a vacuum centrifuge, they were reconstituted in 50 ul ddH₂O for LC-MS/MS analysis. Gut content and fecal samples were weighed, then homogenized in ice-cold IS buffer, centrifuged for 1 min at 500 g and the supernatant was immediately stored at −80 C for analysis. Gut and brain tissue was also frozen for real-time quantitative PCR (RT-qPCR) and metabolite analysis. Quantification of metabolites from in vivo samples was performed as described above (‘LC-MS analysis’).

Results Summary

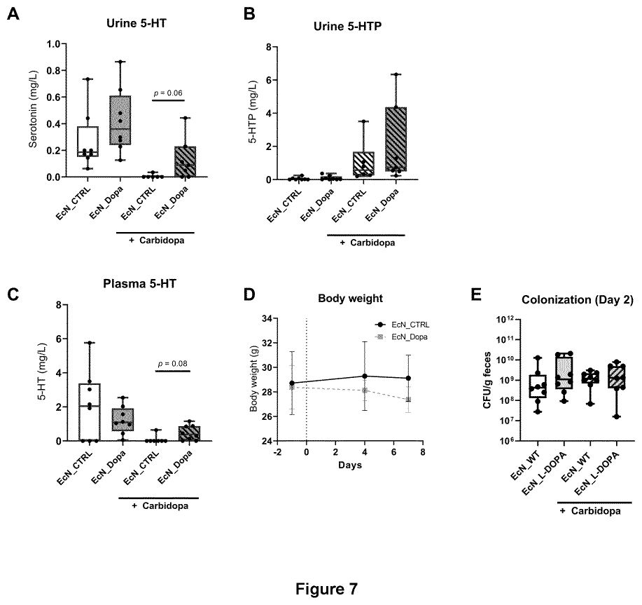

The results are shown in FIG. 7.

Oral delivery of the genetically modified E. coli Nissle strains of the invention and their effect on host physiology was demonstrated in mice. The L-DOPA producing strain was shown to affect metabolite levels in urine and plasma, compared to a non-producing control strain (FIG. 7A-C). The L-DOPA producing strain was also shown to affect body weight in mice (FIG. 7 D). Figure (E) shows Colony forming units (CFU) per grams of feces from mice treated with EcN_WT and EcN_DOPA after 2 days of gavage.

### Exampled 5-7 Relate to Downstream Dopamine Production

General Protocol for Production of Dopamine

Biological triplicates of E. coli Nissle strains harboring the different promoter constructs were grown, using 96 deep-well plates, in 350 μl of M9 minimal media with 0.4% glucose and 0.2% Cas amino acids for 24 hours in a shaking incubator at 37° C. and 250 RPM. Production culture was inoculated with 1:100 inoculum from the preculture in fresh M9 minimal media with 0.4% glucose and 0.2% Cas amino acids. The plate was incubated for 22 hours at 37° C. and 250 RPM. The production culture was then centrifuged at 4500 RPM and supernatants were transferred into a 96 well microtiter plate and stored at −20° C. until HPLC analysis.

### Example 5: Specific Decarboxylases Enhance the Production of Dopamine and Reduce the Production of Side-Products

A panel of L-DOPA decarboxylases was tested in combination with tyrosine hydroxylase. The DDCs were on a different plasmid, called pMK-DDC (FIG. 12 shows the general layout, all the different DDCs were in this format). The two plasmids were co-transformed into EcN_GFP(folET198I) and tested as described below.

The same culture conditions were used as described above, with the only difference that 100 mg/L L-tyrosine was supplemented in the medium. This information is also in the table above.

TABLE 7
L-DOPA Decarboxylases tested

| Name in FIG. 8 | Decarboxylase | Source | Sequence ID |
|---|---|---|---|
| DRO | amino acid | Draconibacterium | WP_038564913.1 |
| | decarboxylase | orientale | |
| NID | amino acid | Nisaea denitrificans | WP_028467075.1 |
| | decarboxylase | | |
| VEM | pyridoxal-dependent | Verrucosispora | WP_013735011.1 |
| | decarboxylase | | |
| CK | Aromatic-L-amino-acid | Candidatus | ABF41161.1 |
| | decarboxylase | Koribacter versatilis | |
| | | Ellin345 | |
| SS | Aromatic-L-amino-acid | Sus scrofa | P80041.2 |
| | decarboxylas | | |
| CR | Aromatic-L-amino-acid | Catharanthus | P17770.1 |
| | decarboxylase | roseus | |
| HS | aromatic-L-amino-acid | Homo sapiens | NP_000781.2 |
| | decarboxylase isoform 1 | | |
| 2833 | aromatic-L-amino-acid | Capsicum annuum | NP_001312016.1 |
| | decarboxylase-like | | |
| 2851 | tryptophan | Oryza sativa | XP_015648701.1 |
| | decarboxylase 1-like | Japonica | |
| 3596 | Tryptophan | Camptotheca | P93082.1 |
| | decarboxylase TDC1 | acuminata | |
| 3597 | tryptophan | Ophiorrhiza pumila | BAC41515.1 |
| | decarboxylase | | |
| EF | tyrosine decarboxylase | Enterococcus | WP_141442151.1 |
| | | faecalis | |
| EFop | tyrosine | Enterococcus | WP_141442151.1 |
| | decarboxylase (codon | faecalis | |
| | optimized) | | |

Detection and quantification of L-DOPA, dopamine, tyrosine, tyramine, phenethylamine, serotonin, tryptamine, tryptophan, and 5-HTP were conducted by liquid chromatography mass spectrometry (LC-MS) measurements on a Dionex UltiMate 3000 UHPLC (Fisher Scientific, San Jose, Calif.) connected to an Orbitrap Fusion Mass Spectrometer (Thermo Fisher Scientific, San Jose, Calif.). The system used an Agilent Zorbax Eclipse Plus C18 2.1×100 mm, 1.8 μm column kept at 35° C. The flow rate was 0.350 mL/min with 0.1% formic acid (A) and 0.1% formic acid in acetonitrile (B) as mobile phase. The gradient started as 5% B and followed a linear gradient to 35% B over 1.5 min. This solvent composition was held for 3.5 min after which it was changed immediately to 95% B and held for 1 min. Finally, the gradient was changed to 5% B until 6 min. The sample (1 uL) was passed on to the MS equipped with a heated electrospray ionization source (HESI) in positive-ion mode with sheath gas set to 60 (a.u.), aux gas to 20 (a.u.) and sweep gas to 2 (a.u.). The cone and probe temperature were 380° C. and 380° C., respectively, and spray voltage was 3500 V. Scan range was 50 to 500 Da and time between scans was 100 ms. Quantification of the compounds was based on calculations from calibration standards analyzed before and after sets of 24 samples. All reagents used were of analytical grade.

Results Summary

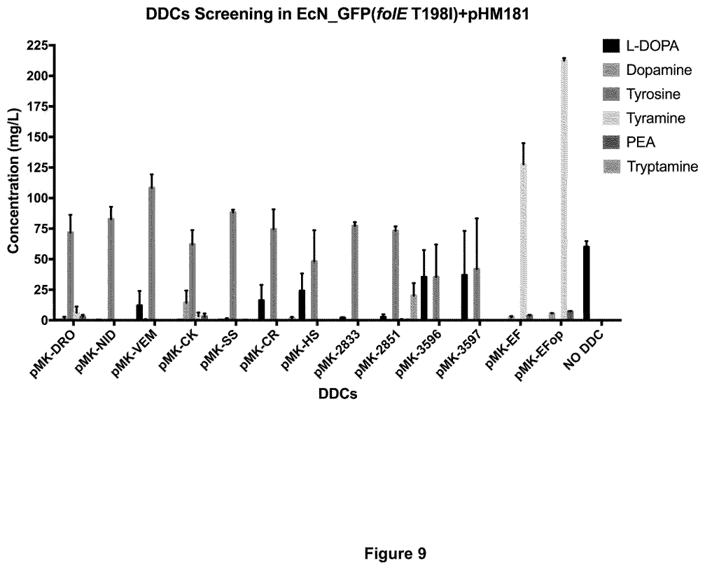

The results are shown in FIG. 9.

The best DDCs that produced measurable amounts of dopamine were: DRO, CK, SS, EF, EFop). These were selected for further testing with variants of the TyrH enzyme as described below in Example 7.

### Example 6: Mutating Tyrosine Hydroxylase for Better Dopamine Production

The truncated tyrosine hydroxylase was used as the background for testing mutations top optimize dopamine production.

A mutation at position 196 in the full length: position 40 in the truncated enzyme was made (position 41 including the start codon).

This is at the following sequence for the truncated enzyme:

(SEQ ID NO.s 6 and 8)
MKSAREDKVPWFPRKVSELDKCHHLVTKFDPDLDLDHPGF[E/L]DQVY
RQRRKLIAEIAFQYKHGEPIPHVEYTAEEIATWKEVYVTLKGLYATHAC
REHLEGFQLLERYCGYREDSIPQLEDVSRFLKERTGFQLRPVAGLLSAR
DFLASLAFRVFQCTQYIRHASSPMHSPEPDCCHELLGHVPMLADRTFAQ
FSQDIGLASLGASDEEIEKLSTVYWFTVEFGLCKQNGELKAYGAGLLSS
YGELLHSLSEEPEVRAFDPDTAAVQPYQDQTYQPVYFVSESFNDAKDKL
RNYASRIQRPFSVKFDPYTLAIDVLDSPHTIQRSLEGVQDELHTLAHAL
SAIS

The same methods as described above (Plasmid construction, Nissle transformation and Production of L-DOPA) were used to test for L-DOPA production.

Results Summary

The results are shown in FIG. 10.

Variation of ser40 of tyrosine hydroxylase (Ser41 with the start codon included) to ser40glu and ser40leu affects production of L-DOPA. The characterized variations surprisingly decrease L-DOPA production yet increase dopamine production. These truncated mutants are SEQ ID NO.s 6 and 8.

### Example 7: Overall Optimization of the Dopamine Pathway for Therapeutic Purposes

Uracil primers containing the codon substitution for ser40 were used to amplify the plasmid containing the TH. The PCR product was purified and USER cloning protocol was followed (described above). The correct construct was later transformed into E. coli Nissle for further production characterization (also described previously).

The mutant was then tested with various decarboxylases to look for the strain which produced the most dopamine and the fewest side products.

The best DDCs were combined with the different versions of the TH (still using a 2 plasmid system, and feeding 100 mg/L L-tyrosine in the medium) and tested for production of dopamine under the same culture conditions, but in the absence of 100 mg/L supplemented Tyrosine. Therefore, all dopamine produced in FIG. 11 is derived from internally produced tyrosine and a small fraction from tyrosine in the supplemented Cas amino acids, which is then converted to L-DOPA, then to Dopamine.

Results Summary

The results are shown in FIG. 11.

Under these conditions, a construct was found (TyrH(Ser40Leu)+SS-DDC) which produces dopamine with a titer of approximately 25 mg/L without any detectable byproducts.

### Examples 8-9 Relate to Further Optimization of the L-DOPA Producing Cells

### Example 8: Further Promoters

A further promoter (Anderson J23101) was tested for driving the expression of cofactor genes.

This promoter is SEQ ID NO. 39 (tttacagctagctcagtcctaggtattatgctagc). Other Anderson promoters that could be used are in SEQ ID NO.s 38 and 40-50.

The strains tested were as follows:

| Strain | Genotype | Plasmid |
|---|---|---|
| 514 | EcN_GFP(ΔfolE | Empty |
| | T198I)ΔTyrR | |
| 519 | EcN_GFP(ΔfolE | Pmic7::ratTH_trunc; Ptrc::phhB |
| | T198I)ΔTyrR | |
| 667 | EcN_GFP(ΔfolE | Pmic7::ratTH_trunc; Ptrc::phhB, |
| | T198I)ΔTyrR | folE(T98I), folM |
| 838 | EcN_GFP(ΔfolE | Pmic7::ratTH_trunc; |
| | T198I)ΔTyrR | PJ23101::phhB, folE(T98I), folM (folE |
| | | and folM codon optimized for E. coli). |

Briefly, 514 is an empty control, 519 does not have overexpression of folE and folM and 838 is the new strain with the codon optimized folE and folM and the Anderson promoter.

Results Summary

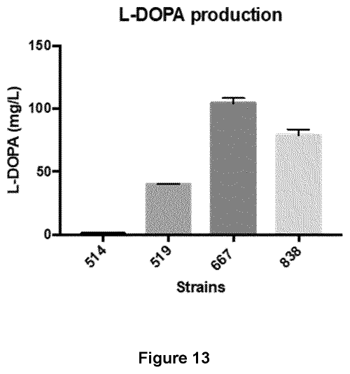

The results are shown in FIG. 13.

Although the 838 strain produced lower amounts of L-DOPA, the Anderson promoter still produces L-DOPA in large amounts. The Anderson promoter is therefore an option for in vivo expression. By varying this promoter sequence, the amount of L-DOPA can be modified further (either up or down).

### Example 9: Integration

Genome integration was carried out using the pOSIP clone integration approach (St-Pierre et al, “One-step Cloning and Chromosomal Integration of DNA”, ACS Synth. Biol. 2013, 2, 9, 537-541).

The integration site used was the att186 integration site.

Results Summary

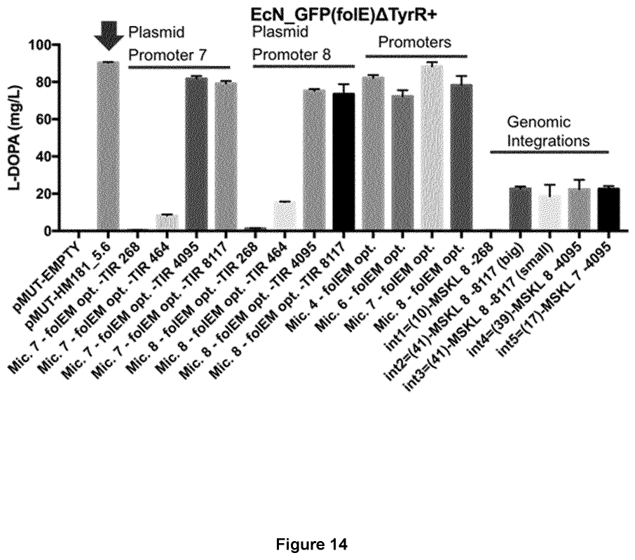

The results are shown in FIG. 14.

The constructs tested are listed on the x-axis:

Column 1=strain 514 (empty plasmid)

All the further columns tested constructs with the codon optimized folE and folM and the new Anderson promoter (J23101). Instead, the promoter driving tyrosine hydroxylase expression was varied along with the RBS.

The constructs on the x axis are listed in the table below.

| | | | | folE, folM |
|---|---|---|---|---|
| | Promoter | RBS | Promoter for | codon |
| Column | for TH | for TH | phhB, folE, folM | optimized |
| pMut- | — | — | — | — |
| EMPTY | | | | |
| pMUT- | TGCTTGACTCGT | atgtggaatttc | Ttgacaattaatcatccggctcgta | NO |
| HM181_5.6 | CGTTCCTCCTAC | acacaggagt | taatg (trc) | |
| | GTGTATAATTGG | aaaa | | |
| Mic. 7 | TGCTTGACTCGT | ATGTGGA | tttacagctagctcagtcctaggtat | Yes |
| folEM opt. | CGTTCCTCCTAC | ATTTCACT | tatgctagc (J23101) | |
| TIR 268 | GTGTATAATTGG | CAGGCGG | | |
| | | AAAA | | |
| Mic. 7 | TGCTTGACTCGT | ATGTGGA | tttacagctagctcagtcctaggtat | Yes |
| folEM opt. | CGTTCCTCCTAC | ATTTCACT | tatgctagc (J23101) | |
| TIR 464 | GTGTATAATTGG | CAGGTGG | | |
| | | AAAA | | |
| Mic. 7 | TGCTTGACTCGT | ATGTGGA | tttacagctagctcagtcctaggtat | Yes |
| folEM opt. | CGTTCCTCCTAC | ATTTCACT | tatgctagc (J23101) | |
| TIR 4095 | GTGTATAATTGG | CTGGAGG | | |
| | | AAAA | | |
| Mic. 7 | TGCTTGACTCGT | ATGTGGA | tttacagctagctcagtcctaggtat | Yes |
| folEM opt. | CGTTCCTCCTAC | ATTTCACT | tatgctagc (J23101) | |
| TIR 8117 | GTGTATAATTGG | CAGGAGG | | |
| | | AAAA | | |
| Mic. 8 | TGCTTGACTCGT | ATGTGGA | tttacagctagctcagtcctaggtat | Yes |
| folEM opt. | CGTTATCCTACGT | ATTTCACT | tatgctagc (J23101) | |
| TIR 268 | GTATAATTGGC | CAGGCGG | | |
| | | AAAA | | |
| Mic. 8 | TGCTTGACTCGT | ATGTGGA | tttacagctagctcagtcctaggtat | Yes |
| folEM opt. | CGTTATCCTACGT | ATTTCACT | tatgctagc (J23101) | |
| TIR 464 | GTATAATTGGC | CAGGTGG | | |
| | | AAAA | | |
| Mic. 8 | TGCTTGACTCGT | ATGTGGA | tttacagctagctcagtcctaggtat | Yes |
| folEM opt. | CGTTATCCTACGT | ATTTCACT | tatgctagc (J23101) | |
| TIR 4095 | GTATAATTGGC | CTGGAGG | | |
| | | AAAA | | |
| Mic. 8 | TGCTTGACTCGT | ATGTGGA | tttacagctagctcagtcctaggtat | Yes |
| folEM opt. | CGTTATCCTACGT | ATTTCACT | tatgctagc (J23101) | |
| TIR 8117 | GTATAATTGGC | CAGGAGG | | |
| | | AAAA | | |
| Mic. 4 | GGATTGACAATAT | atgtggaatttc | tttacagctagctcagtcctaggtat | Yes |
| folEM opt. | AGGCTGGAGCTT | acacaggagt | tatgctagc (J23101) | |
| | CTAGTATTGAA | aaaa | | |
| Mic. 6 | TGCTGGACTCGT | atgtggaatttc | tttacagctagctcagtcctaggtat | Yes |
| folEM opt. | CGTAATCCTGCG | acacaggagt | tatgctagc (J23101) | |
| | TGTATAATTGGC | aaaa | | |
| Mic. 7 | TGCTTGACTCGT | atgtggaatttc | tttacagctagctcagtcctaggtat | Yes |
| folEM opt. | CGTTCCTCCTAC | acacaggagt | tatgctagc (J23101) | |
| | GTGTATAATTGG | aaaa | | |
| Mic. 8 | TGCTTGACTCGT | atgtggaatttc | tttacagctagctcagtcctaggtat | Yes |
| folEM opt. | CGTTATCCTACGT | acacaggagt | tatgctagc (J23101) | |
| | GTATAATTGGC | aaaa | | |
| Int1 = (10)- | TGCTTGACTCGT | ATGTGGA | tttacagctagctcagtcctaggtat | Yes |
| MSKL 8- | CGTTATCCTACGT | ATTTCACT | tatgctagc (J23101) | |
| 268 | GTATAATTGGC | CAGGCGG | | |
| | | AAAA | | |
| Int2 = (41)- | TGCTTGACTCGT | ATGTGGA | tttacagctagctcagtcctaggtat | Yes |
| MSKL 8- | CGTTATCCTACGT | ATTTCACT | tatgctagc (J23101) | |
| 8117(big) | GTATAATTGGC | CAGGAGG | | |
| | | AAAA | | |
| Int3 = (41)- | TGCTTGACTCGT | ATGTGGA | tttacagctagctcagtcctaggtat | Yes |
| MSKL 8- | CGTTATCCTACGT | ATTTCACT | tatgctagc (J23101) | |
| 8117(small) | GTATAATTGGC | CAGGAGG | | |
| | | AAAA | | |
| Int4 = (39)- | TGCTTGACTCGT | ATGTGGA | tttacagctagctcagtcctaggtat | Yes |
| MSKL 8- | CGTTATCCTACGT | ATTTCACT | tatgctagc (J23101) | |
| 4095 | GTATAATTGGC | CTGGAGG | | |
| | | AAAA | | |
| Int5 = (17)- | TGCTTGACTCGT | ATGTGGA | tttacagctagctcagtcctaggtat | Yes |
| MSKL 8- | CGTTATCCTACGT | ATTTCACT | tatgctagc (J23101) | |
| 4095 | GTATAATTGGC | CTGGAGG | | |
| | | AAAA | | |

The last 5 columns (labelled “Int”) integrate the constructs into the genome.

For example MSKL 8-8117 means the promoter used was MSKL 8 with a RBS with TIR of 8117. The terms “big” and “small” refer to the colony size only. Both types produced L-DOPA.

Varying the RBS did not alter L-DOPA production. The best construct as denoted by the arrow in FIG. 14. This is strain 667 (Mic promoter 7 driving TH and trc promoter driving the expression of phhB, folE mutant and folM), see example 8 above.

### Example 10: Comparison with Bacterial Pathway Enzymes

Biological triplicates of E. coli Nissle strains harboring the different tyrosine hydroxylases or bacterial enzymes constructs were grown, using 96 deep-well plates, in 350 μl of M9 minimal media with 0.4% glucose for 24 hours in a shaking incubator at 37° C. and 250 RPM. Production culture was inoculated with 1:100 inoculum from the preculture in fresh M9 minimal media with 0.4% glucose. The plate was incubated for 22 hours at 37° C. and 250 RPM. The production culture was then centrifuged at 4500 RPM and supernatants were transferred into a 96 well microtiter plate and stored at −20° C. until HPLC analysis.

| Strain | Genotype | Plasmid |
|---|---|---|
| 519 | EcN_GFP(ΔfolE | Pmic7::ratTH_trunc; Ptrc::phhB |
| | T198I)ΔTyrR | |
| 838 | EcN_GFP(ΔfolE | Pmic7::ratTH_trunc; |
| | T198I)ΔTyrR | PJ23101::phhB, folE(T98I), folM (folE |
| | | and folM codon optimized for E. coli). |
| Rat Full | EcN_GFP(ΔfolE | Pmic7::ratTH_full; |
| | T198I)ΔTyrR | PJ23101::phhB, folE(T98I), folM (folE |
| | | and folM codon optimized for E. coli). |
| HpaBC | EcN_GFP(ΔfolE | Pmic7::hpaBC |
| | T198I)ΔTyrR | |

Results Summary

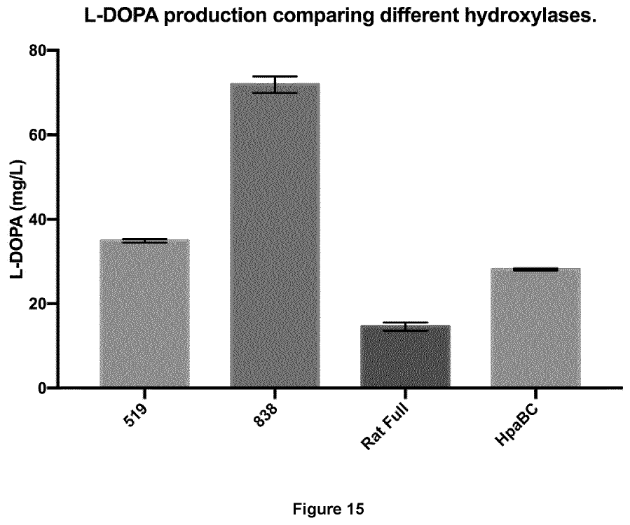

The results are shown in FIG. 15.

The results show that in the same genetic background and under the same conditions, the 838 and 519 strains produced more L-DOPA compared to the bacterial enzyme pathway which is based on E. coli native enzymes (HpaBC). A further advantage of TyrH, is that it is highly specific towards tyrosine unlike the bacterial enzymes (monooxygenases like hpaBC) that tend to be promiscuous in their substrate preference.

Additionally, we show that expressing the full length tyrosine hydroxylase from rat (codon optimized) E. coli Nissle is able to produce L-DOPA.

### Example 11: In Vivo Production of L-DOPA in Plasma

Gavage Preparation:

A single colony of each bacterial strain was grown in 50 ml of 2×YT for at least 16 hours at 37° C. and 250 RPM in a shaking incubator. Cultures were then washed with PBS and adjusted to contain 0.5×10¹⁰ CFU/ml.

Animals and Experiments:

Male Sprague Dawley rats were acclimatized for 1 week before randomized grouping (4 per group). 5 g/L of Streptomycin in drinking water was started 3 days prior of the gavage regime and was maintained throughout the experiment. Animals were gavaged 2 ml of 10¹⁰ CFU daily for 3 days (days 0-2). On day 3, animals were given 25 mg/Kg (IP) of carbidopa 1 hour prior the gavage containing 4×10¹⁰ CFU/ml and tyrosine (50 mg/Kg). Animals were sacrificed on day 4 and jugular blood was collected after decapitation. CFUs were determined from fecal and gut content samples.

Extraction of Plasma L-DOPA:

L-DOPA from plasma was extracted using an Ostro Protein Precipitation & Phospholipid Removal Plate following manufacturer's guidelines (100 μl of plasma), samples were dried using a speedvac with no heating and resuspended in MQ water containing 0.1% Ascorbic acid and formic acid. C-13 L-DOPA internal standard was spiked before the extraction method to account for any loss throughout the procedure. Internal standard solution also contained 0.1% ascorbic acid.

Lc-Ms/Ms Quantification:

The LC-MS/MS analysis was performed on a Vanquish Duo UHPLC binary system (Thermo Fisher Scientific, USA) coupled to the IDX-Orbitrap Mass Spectrometer (Thermo Fisher Scientific, USA). The analytes were separated using a Waters ACQUITY BEH C18 (10 cm×2.1 mm, 1.7 μm) column equipped with an ACQUITY BEH C18 guard column kept at 40 C. The mobile phases consisted of MilliQ© water+0.1% formic acid (A) and acetonitrile+0.1% formic acid (B). The initial composition was 2% B held for 0.8 min, followed by a linear gradient till 5% in 3.3 min, and to 100% B in 10 min held for 1 min before going back to initial conditions. Re-equilibration time was 2.7 min. The flow rate was set at 0.35 mL/min. The MS measurements were done in positive and negative-heated electrospray ionization (HESI) mode with a voltage of 3500 V and 2500 V respectively acquiring in full MS/MS spectra (Data dependent Acquisition-driven MS/MS) in the m/z range of 70-1000. The acquired data were processed using QuanBrowser from the Xcalibur software v 4.4 (Thermo Fisher Scientific, USA).

Results Summary

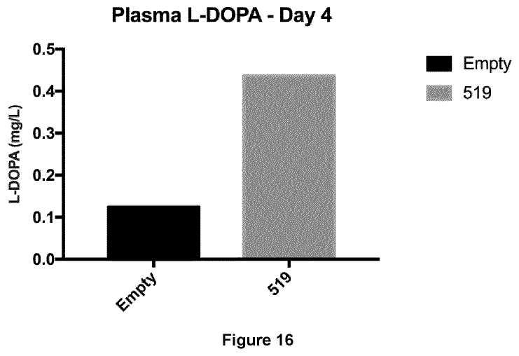

The results are shown in FIG. 16.

These results show that L-DOPA plasma levels were substantially increased in rats that were treated with a EcN with L-DOPA production capabilities (a: 0.511) (strain 519) compared to an empty strain, which does not produce any L-DOPA (a: 0.034).

### Example 12: Additional Copy Numbers of Tyrosine Hydroxylase

Methods:

The same cultivation methods as described above were used to test expression of L-DOPA from the following strains:
Plasmid: strain 426 (EcN_GFP (folE mut) ΔtyrR(KO)+pMUT-HM181) as described above
Integration+plasmid: Integrated strain (also as described above)+pMUT-HM181

L-DOPA was quantified using HPLC as described above.

Results Summary

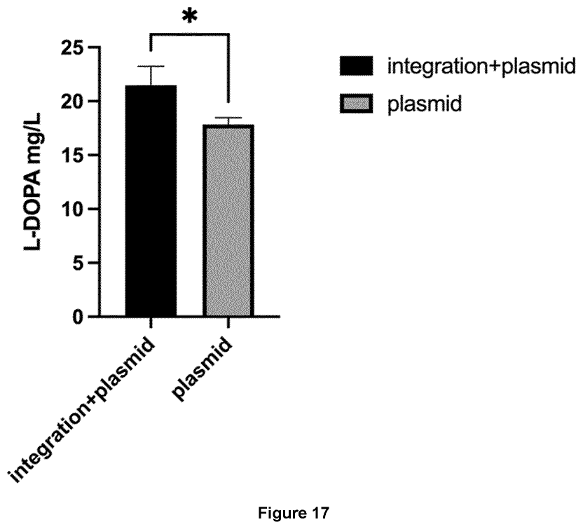

The results are shown in FIG. 17.

The results indicate that an extra expression component (where the integrated strain ALSO has the plasmid) boosts L-DOPA production (P=0.0273).

The results support that multiple copies in the chromosome, plasmid or combined (chromosome and plasmid) should increase microbial L-DOPA production capabilities, allowing the titration of L-DOPA in vivo.

1. A microbial cell comprising: a recombinant nucleic acid encoding a eukaryotic tyrosine hydroxylase, for use as a medicament.

2. The microbial cell for use of claim 1, for use in a method of treating Parkinson's disease.

3. The microbial cell for use of claim 1, for use in a method of treating a dopamine-related disorder.

4. A microbial cell comprising a recombinant nucleic acid encoding a eukaryotic tyrosine hydroxylase, wherein the microbial cell is a therapeutic microbial cell, optionally E. coli Nissle.

5. A pharmaceutical formulation comprising a microbial cell wherein the microbial cell comprises a recombinant nucleic acid encoding a eukaryotic tyrosine hydroxylase.

6. The microbial cell for use, pharmaceutical formulation or microbial cell of claims 1-5, wherein the microbial cell:
a) additionally comprises a nucleic acid encoding a compound which inhibits an L-DOPA metabolizing bacteria; or
b) is co-administered with:
i) a compound which inhibits an L-DOPA-metabolizing bacteria; or
ii) a further microbial cell which produces a compound which inhibits an L-DOPA-metabolizing bacteria.

7. The microbial cell for use, pharmaceutical formulation or microbial cell of claims 1-6, wherein the microbial cell additionally comprises:
a) a recombinant nucleic acid encoding a 4a-hydroxytetrahydrobiopterin dehydratase; and/or
b) an σ⁷⁰ promoter.

8. A microbial cell comprising:
a) a recombinant nucleic acid encoding a eukaryotic tyrosine hydroxylase; and
b) a recombinant nucleic acid encoding an enzyme having L-DOPA decarboxylase activity.

9. The microbial cell of claim 8, wherein the tyrosine hydroxylase is a mutant enzyme wherein:
a) the mutant tyrosine hydroxylase does not comprise a functional regulatory domain; and/or
b) the mutant tyrosine hydroxylase comprises a mutation in the catalytic domain.

10. The microbial cell of claim 9b, wherein the mutation corresponds to:
a) any one of amino acids 177-198 of SEQ ID NO. 2, optionally wherein the mutation is at an amino acid corresponding to amino acid 196 of SEQ ID NO. 2, optionally wherein the mutation is Ser196Glu or Ser196Leu; or
b) any one of amino acids 22-43 of SEQ ID NO. 4, optionally wherein the mutation is at an amino acid corresponding to amino acid 41 of SEQ ID NO. 4, optionally wherein the mutation is Ser41Glu or Ser41Leu.

11. The microbial cell of claims 8-10, wherein the L-DOPA decarboxylase enzyme belongs to any one of the following:
a) EC:4.1.1.28, optionally wherein the enzyme has at least 70% sequence identity to SEQ ID NO.s 18, 20 or 22;
b) EC:4.1.1.105, optionally wherein the enzyme has at least 70% sequence identity to SEQ ID NO.s 20 or 22;
c) EC:4.1.1.25 optionally wherein the enzyme has at least 70% sequence identity to SEQ ID NO. 25.

12. The microbial cell of claim 11, wherein the enzyme having L-DOPA decarboxylase activity has at least 70% sequence identity to SEQ ID NO. 18.

13. A pharmaceutical formulation comprising any of the microbial cells of claims 8-12.

14. The microbial cell of claims 8-12 or the pharmaceutical formulation of claim 13 for use as a medicament.

15. The microbial cell of claim 14 for use in a method of treating a dopamine-related disorder.

16. The microbial cell, microbial cell for use or pharmaceutical formulation of any of the preceding claims, wherein the tyrosine hydroxylase belongs to EC 1.14.16.2.

17. The microbial cell, microbial cell for use or pharmaceutical formulation of any of the preceding claims, wherein the tyrosine hydroxylase does not comprise the regulatory domain.

18. The microbial cell, microbial cell for use or pharmaceutical formulation of claim 17, wherein the tyrosine hydroxylase comprises the catalytic domain and the tetramerization domain of the eukaryotic tyrosine hydroxylase enzyme, optionally wherein the tyrosine hydroxylase has at least 70% sequence identity to SEQ ID NO. 4.

19. The microbial cell, microbial cell for use or pharmaceutical formulation of any of the preceding claims, wherein the microbial cell additionally comprises a nucleic acid encoding a mutant GTP cyclohydrolase I, the mutant GTP cyclohydrolase I having at least 70% sequence identity to SEQ ID NO. 10, and comprising one or more mutations wherein the mutant provides for an increased hydroxylation activity of the tyrosine hydroxylase.

20. The microbial cell, microbial cell for use or pharmaceutical formulation of claim 19 wherein the GTP cyclohydrolase I mutant is at a position corresponding to amino acid 198 of SEQ ID NO. 10.

21. The microbial cell, microbial cell for use or pharmaceutical formulation of any of the preceding claims further comprising:
a) a nucleic acid encoding a 4a-hydroxytetrahydrobiopterin dehydratase (phhB), optionally wherein the phhB belongs to EC 4.2.1.96 and/or has at least 70% sequence identity to SEQ ID NO. 14; and/or
b) a nucleic acid encoding a dihydromonapterin reductase (FolM), optionally wherein the FolM has at least 70% sequence identity to SEQ ID NO. 12.

22. The microbial cell, or the microbial cell for use of any of any of the preceding claims, wherein the nucleic acid(s) is integrated into the genome of the microbial cell.

23. A recombinant expression plasmid comprising:
a) a recombinant nucleic acid encoding a eukaryotic tyrosine hydroxylase;
and any one or more of the following:
b) i) a recombinant nucleic acid encoding a 4a-hydroxytetrahydrobiopterin dehydratase; and/or
ii) an σ⁷⁰ promoter; and/or
iii) a recombinant nucleic acid encoding a compound which inhibits an L-DOPA metabolizing bacteria.

24. A recombinant expression plasmid comprising:
a) a recombinant nucleic acid encoding a eukaryotic tyrosine hydroxylase; and
b) a recombinant nucleic acid encoding an enzyme having L-DOPA decarboxylase activity.

25. A mutant eukaryotic tyrosine hydroxylase wherein the mutation is at an amino acid corresponding to:
a) any one of amino acids 177-198 of SEQ ID NO. 2, optionally wherein the mutation is at an amino acid corresponding to amino acid 196 of SEQ ID NO. 2, optionally wherein the mutation is Ser196Glu or Ser196Leu; or
b) any one of amino acids 22-43 of SEQ ID NO. 4, optionally wherein the mutation is at an amino acid corresponding to amino acid 41 of SEQ ID NO. 4, optionally wherein the mutation is Ser41Glu or Ser41Leu.


SEQUENCE LISTING (56 sequences)

SEQ ID NO: 1 - DNA, 1497 bp, Rattus norvegicus
atgccaacacctagtgctccgagtccccaacctaaggggtttcgtcgcgcggtaagcgaacaagacgcaaaacaggctgaggcagttactagtccacgttttatcggccgccgtcaatcgttaattgaagatgcgcgcaaggagcgcgaggctgcggcagccgccgcagctgcagctgtagcgagtagcgaaccggggaatcctctggaggctgttgttttcgaagagcgtgacggtaacgcggtactgaatttgttattcagtcttcgcggcactaagcccagcagtctttcccgtgccgttaaagtattcgagactttcgaggccaagatccatcatttggagacgcgtcctgcacaacgtccgttagcagggtcgccacatttggaatacttcgtccgcttcgaggtaccaagcggcgatcttgccgccttattgagtagcgtgcgtcgcgtctcggatgatgtgcgtagcgcacgtgaagataaagttccgtggtttccgcgtaaagtgagcgaactggataaatgtcatcatctggtgaccaaatttgatcctgatctggatctggaccatccgggttttagcgatcaggtttatcgtcagcgtcgtaaactgattgcagaaattgcctttcagtataaacatggtgaaccgattccgcacgttgaatataccgcagaagaaattgcaacctggaaagaagtttacgttaccctgaaaggtctgtatgcaacccatgcatgtcgtgaacatctggaaggttttcagctgctggaacgttattgtggttatcgcgaagatagcattccgcagctggaagatgttagccgttttctgaaagaacgtaccggttttcaactgcgtccggttgcaggtctgctgagtgcacgtgattttctggcaagcctggcatttcgtgtttttcagtgtacccagtatattcgtcatgccagcagcccgatgcatagtccggaaccggattgttgtcatgaactgctgggtcatgttccgatgctggcagatcgtacctttgcacagtttagccaggatattggtctggcatcactgggtgcaagtgatgaagaaatcgaaaaactgagcaccgtgtattggtttaccgttgaatttggtctgtgcaaacagaatggtgaactgaaagcatatggtgccggtctgctgtcaagctatggcgaactgctgcatagcctgagcgaagaaccggaagttcgtgcatttgatccggataccgcagccgttcagccgtatcaggaccagacctatcagccggtttattttgttagcgaaagcttcaatgacgccaaagataaactgcgtaattatgcaagccgtattcagcgtccgtttagcgttaaattcgatccgtataccctggcaattgatgttctggattcaccgcataccattcagcgtagcctggaaggtgtgcaggatgaactgcatacactggcacatgcactgagcgcaattagctaa

SEQ ID NO: 2 - protein, 498 aa, Rattus norvegicus
MPTPSAPSPQPKGFRRAVSEQDAKQAEAVTSPRFIGRRQSLIEDARKEREAAAAAAAAAVASSEPGNPLEAVVFEERDGNAVLNLLFSLRGTKPSSLSRAVKVFETFEAKIHHLETRPAQRPLAGSPHLEYFVRFEVPSGDLAALLSSVRRVSDDVRSAREDKVPWFPRKVSELDKCHHLVTKFDPDLDLDHPGFSDQVYRQRRKLIAEIAFQYKHGEPIPHVEYTAEEIATWKEVYVTLKGLYATHACREHLEGFQLLERYCGYREDSIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTVYWFTVEFGLCKQNGELKAYGAGLLSSYGELLHSLSEEPEVRAFDPDTAAVQPYQDQTYQPVYFVSESFNDAKDKLRNYASRIQRPFSVKFDPYTLAIDVLDSPHTIQRSLEGVQDELHTLAHALSAIS

SEQ ID NO: 3 - DNA, 1032 bp, Artificial Sequence
  Truncated rat Tyrosine Hydroxylase
atgaaaagcgcacgtgaagataaagttccgtggtttccgcgtaaagtgagcgaactggataaatgtcatcatctggtgaccaaatttgatcctgatctggatctggaccatccgggttttagcgatcaggtttatcgtcagcgtcgtaaactgattgcagaaattgcctttcagtataaacatggtgaaccgattccgcacgttgaatataccgcagaagaaattgcaacctggaaagaagtttacgttaccctgaaaggtctgtatgcaacccatgcatgtcgtgaacatctggaaggttttcagctgctggaacgttattgtggttatcgcgaagatagcattccgcagctggaagatgttagccgttttctgaaagaacgtaccggttttcaactgcgtccggttgcaggtctgctgagtgcacgtgattttctggcaagcctggcatttcgtgtttttcagtgtacccagtatattcgtcatgccagcagcccgatgcatagtccggaaccggattgttgtcatgaactgctgggtcatgttccgatgctggcagatcgtacctttgcacagtttagccaggatattggtctggcatcactgggtgcaagtgatgaagaaatcgaaaaactgagcaccgtgtattggtttaccgttgaatttggtctgtgcaaacagaatggtgaactgaaagcatatggtgccggtctgctgtcaagctatggcgaactgctgcatagcctgagcgaagaaccggaagttcgtgcatttgatccggataccgcagccgttcagccgtatcaggaccagacctatcagccggtttattttgttagcgaaagcttcaatgacgccaaagataaactgcgtaattatgcaagccgtattcagcgtccgtttagcgttaaattcgatccgtataccctggcaattgatgttctggattcaccgcataccattcagcgtagcctggaaggtgtgcaggatgaactgcatacactggcacatgcactgagcgcaattagctaa

SEQ ID NO: 4 - protein, 343 aa, Artificial Sequence
  Truncated rat Tyrosine Hydroxylase
MKSAREDKVPWFPRKVSELDKCHHLVTKFDPDLDLDHPGFSDQVYRQRRKLIAEIAFQYKHGEPIPHVEYTAEEIATWKEVYVTLKGLYATHACREHLEGFQLLERYCGYREDSIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTVYWFTVEFGLCKQNGELKAYGAGLLSSYGELLHSLSEEPEVRAFDPDTAAVQPYQDQTYQPVYFVSESFNDAKDKLRNYASRIQRPFSVKFDPYTLAIDVLDSPHTIQRSLEGVQDELHTLAHALSAIS

SEQ ID NO: 5 - DNA, 1032 bp, Artificial Sequence
  Truncated rat Tyrosine Hydroxylase SER196GLU
atgaaaagcgcacgtgaagataaagttccgtggtttccgcgtaaagtgagcgaactggataaatgtcatcatctggtgaccaaatttgatcctgatctggatctggaccatccgggttttgaagatcaggtttatcgtcagcgtcgtaaactgattgcagaaattgcctttcagtataaacatggtgaaccgattccgcacgttgaatataccgcagaagaaattgcaacctggaaagaagtttacgttaccctgaaaggtctgtatgcaacccatgcatgtcgtgaacatctggaaggttttcagctgctggaacgttattgtggttatcgcgaagatagcattccgcagctggaagatgttagccgttttctgaaagaacgtaccggttttcaactgcgtccggttgcaggtctgctgagtgcacgtgattttctggcaagcctggcatttcgtgtttttcagtgtacccagtatattcgtcatgccagcagcccgatgcatagtccggaaccggattgttgtcatgaactgctgggtcatgttccgatgctggcagatcgtacctttgcacagtttagccaggatattggtctggcatcactgggtgcaagtgatgaagaaatcgaaaaactgagcaccgtgtattggtttaccgttgaatttggtctgtgcaaacagaatggtgaactgaaagcatatggtgccggtctgctgtcaagctatggcgaactgctgcatagcctgagcgaagaaccggaagttcgtgcatttgatccggataccgcagccgttcagccgtatcaggaccagacctatcagccggtttattttgttagcgaaagcttcaatgacgccaaagataaactgcgtaattatgcaagccgtattcagcgtccgtttagcgttaaattcgatccgtataccctggcaattgatgttctggattcaccgcataccattcagcgtagcctggaaggtgtgcaggatgaactgcatacactggcacatgcactgagcgcaattagctaa

SEQ ID NO: 6 - protein, 343 aa, Artificial Sequence
  Truncated rat Tyrosine Hydroxylase SER196GLU
MKSAREDKVPWFPRKVSELDKCHHLVTKFDPDLDLDHPGFEDQVYRQRRKLIAEIAFQYKHGEPIPHVEYTAEEIATWKEVYVTLKGLYATHACREHLEGFQLLERYCGYREDSIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTVYWFTVEFGLCKQNGELKAYGAGLLSSYGELLHSLSEEPEVRAFDPDTAAVQPYQDQTYQPVYFVSESFNDAKDKLRNYASRIQRPFSVKFDPYTLAIDVLDSPHTIQRSLEGVQDELHTLAHALSAIS

SEQ ID NO: 7 - DNA, 1032 bp, Artificial Sequence
  Truncated rat Tyrosine Hydroxylase SER196LEU
atgaaaagcgcacgtgaagataaagttccgtggtttccgcgtaaagtgagcgaactggataaatgtcatcatctggtgaccaaatttgatcctgatctggatctggaccatccgggttttctggatcaggtttatcgtcagcgtcgtaaactgattgcagaaattgcctttcagtataaacatggtgaaccgattccgcacgttgaatataccgcagaagaaattgcaacctggaaagaagtttacgttaccctgaaaggtctgtatgcaacccatgcatgtcgtgaacatctggaaggttttcagctgctggaacgttattgtggttatcgcgaagatagcattccgcagctggaagatgttagccgttttctgaaagaacgtaccggttttcaactgcgtccggttgcaggtctgctgagtgcacgtgattttctggcaagcctggcatttcgtgtttttcagtgtacccagtatattcgtcatgccagcagcccgatgcatagtccggaaccggattgttgtcatgaactgctgggtcatgttccgatgctggcagatcgtacctttgcacagtttagccaggatattggtctggcatcactgggtgcaagtgatgaagaaatcgaaaaactgagcaccgtgtattggtttaccgttgaatttggtctgtgcaaacagaatggtgaactgaaagcatatggtgccggtctgctgtcaagctatggcgaactgctgcatagcctgagcgaagaaccggaagttcgtgcatttgatccggataccgcagccgttcagccgtatcaggaccagacctatcagccggtttattttgttagcgaaagcttcaatgacgccaaagataaactgcgtaattatgcaagccgtattcagcgtccgtttagcgttaaattcgatccgtataccctggcaattgatgttctggattcaccgcataccattcagcgtagcctggaaggtgtgcaggatgaactgcatacactggcacatgcactgagcgcaattagctaa

SEQ ID NO: 8 - protein, 343 aa, Artificial Sequence
  Truncated rat Tyrosine Hydroxylase SER196LEU
MKSAREDKVPWFPRKVSELDKCHHLVTKFDPDLDLDHPGFLDQVYRQRRKLIAEIAFQYKHGEPIPHVEYTAEEIATWKEVYVTLKGLYATHACREHLEGFQLLERYCGYREDSIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTVYWFTVEFGLCKQNGELKAYGAGLLSSYGELLHSLSEEPEVRAFDPDTAAVQPYQDQTYQPVYFVSESFNDAKDKLRNYASRIQRPFSVKFDPYTLAIDVLDSPHTIQRSLEGVQDELHTLAHALSAIS

SEQ ID NO: 9 - DNA, 669 bp, Artificial Sequence
  FolE
atgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtcatcggtctgtcaaaaattaaccgtattgtgcagttctttgcccagcgtccgcaggtacaagaacgtctgacgcagcaaattctcattgcgctacaaacgctgctgggcaccaataacgtggctgtctctatcgacgcggtgcattactgcgtgaaggcgcgtggcatccgcgatgcaaccagtgcaaccacaacgacctctcttggtggattgttcaaatccagtcagaatacgcgccacgagtttctgcgcgctgtgcgtcatcacaactga

SEQ ID NO: 10 - protein, 222 aa, Artificial Sequence
  FolE
MPSLSKEAALVHEALVARGLETPLRPPVHEMDNETRKSLIAGHMTEIMQLLNLDLADDSLMETPHRIAKMYVDEIFSGLDYANFPKITLIENKMKVDEMVTVRDITLTSTCEHHFVTIDGKATVAYIPKDSVIGLSKINRIVQFFAQRPQVQERLTQQILIALQTLLGTNNVAVSIDAVHYCVKARGIRDATSATTTTSLGGLFKSSQNTRHEFLRAVRHHN

SEQ ID NO: 11 - DNA, 723 bp, Artificial Sequence
  folM
atgggtaaaacccagtccttgccaatattaattactggcggaggtcgccgcatcggcctcgccctcgcatggcatttcattaatcaaaagcaaccggtgattgtcagctatcggacgcactatcccgccattgatggactgattaaggcaggtgcgcagtgtattcaggctgatttttcgaccaacgacggtgtgatggcgtttgccgatgaagtactaaaaagcacccatggtctgcgtgctattttgcataacgccagcgcgtggatggcggaaaaaccgggagcgccactgaccgacgtactggcttgcatgatgcagatccacgttaataccccatacctgctcaaccatgctctggaaagattactgcgtgggcacggacacgccgccagcgatatcattcactttaccgattatgtggtggagcgcggtagcgacaaacatattgcgtatgctgcaagcaaagcggcactggataatatgacccgctcgtttgcccgcaagctggcaccggaagtgaaagtgaattctattgcgccatcgctgatcctgtttaatgaacatgatgatgctgaatatcgacaacaggcgctgaataaatcactgatgaaaaccgcgcctggcgagaaagaagtgatcgacctggtcgattacttacttaccagctgctttgtcaccggacgcagtctcccacttgatggcggtcgtcatctgcgttaa

SEQ ID NO: 12 - protein, 240 aa, Artificial Sequence
  folM
MGKTQSLPILITGGGRRIGLALAWHFINQKQPVIVSYRTHYPAIDGLIKAGAQCIQADFSTNDGVMAFADEVLKSTHGLRAILHNASAWMAEKPGAPLTDVLACMMQIHVNTPYLLNHALERLLRGHGHAASDIIHFTDYVVERGSDKHIAYAASKAALDNMTRSFARKLAPEVKVNSIAPSLILFNEHDDAEYRQQALNKSLMKTAPGEKEVIDLVDYLLTSCFVTGRSLPLDGGRHLR

SEQ ID NO: 13 - DNA, 342 bp, Artificial Sequence
  phhB
atgacaggagtaaaaatgaatctgctggaactgagctgtacaccgcagcatggtcagagtccgctggcagataataccgtgaaacagctgctgagcaccctgccggattgggaaatcgttggtattgaactgcgtaaaacctatcgtttcgccaactatcatgaaaccatggcatttgttaatgcactggcatggattgccaatcaagaagatcatcatccggatatgtccgtgcattataatcgtgcagttgtgaacttcagcacccatgatgcaggtggtctgaccctgaatgacttcatctgtgcagcgaaaaccgaagcactgtttcgtcgtccgtaa

SEQ ID NO: 14 - protein, 113 aa, Artificial Sequence
  phhB
MTGVKMNLLELSCTPQHGQSPLADNTVKQLLSTLPDWEIVGIELRKTYRFANYHETMAFVNALAWIANQEDHHPDMSVHYNRAVVNFSTHDAGGLTLNDFICAAKTEALFRRP

SEQ ID NO: 15 - DNA, 363 bp, Artificial Sequence
  FolX
atggcacaacctgccgctattattcgtataaagaaccttcgtttgcgtacgtttatcggaattaaggaagaagaaattaacaaccgtcaggatattgttatcaatgtgactatccactaccccgccgataaagcgcgcactagcgaagatatcaacgatgcgctgaattatcgcaccgtaacgaaaaacattattcagcatgtagagaataaccgtttctctttgctggaaaaattaactcaggatgtgctcgatatcgcacgtgaacatcactgggtgacgtatgctgaagtggagatcgataaactgcacgcgctgcgctacgccgattcggtatccatgaccttaagctggcagcgttaa

SEQ ID NO: 16 - protein, 120 aa, Artificial Sequence
  FolX
MAQPAAIIRIKNLRLRTFIGIKEEEINNRQDIVINVTIHYPADKARTSEDINDALNYRTVTKNIIQHVENNRFSLLEKLTQDVLDIAREHHWVTYAEVEIDKLHALRYADSVSMTLSWQR

SEQ ID NO: 17 - DNA, 1461 bp, Sus scrofa
atgaacgcaagcgattttcgtcgtcgtggtaaagaaatggttgattatatggccgattatctggaaggtattgaaggtcgtcaggtttatccggatgttcagcctggttatctgcgtccgctgattccggcaaccgcaccgcaagaaccggatacctttgaagatattctgcaggatgtggaaaaaatcattatgcctggtgttacccattggcatagcccgtatttctttgcatattttccgaccgcaagcagctatccggcaatgctggcagatatgctgtgtggtgcaattggttgtattggttttagctgggcagcaagtccggcatgtaccgaactggaaaccgttatgatggattggctgggtaaaatgctgcagctgccggaagcatttctggcaggcgaagccggtgaaggtggtggtgttattcagggtagcgcaagcgaagcaaccctggttgcactgctggcagcacgtaccaaagttgttcgtcgtctgcaggcagcatcaccgggtctgacccagggtgcagttctggaaaaactggttgcatatgccagcgatcaggcacatagcagcgttgaacgtgcaggtctgattggtggtgtgaaactgaaagcaattccgagtgatggtaaatttgcaatgcgtgcaagtgcactgcaagaagcactggaacgcgataaagcagccggtctgattccgttttttgttgttgcgaccctgggtacaaccagctgttgcagctttgataatctgctggaagttggtccgatttgccatgaagaggatatttggctgcatgttgatgcagcatatgcaggtagcgcatttatttgtccggaatttcgtcatctgctgaatggtgttgaatttgccgatagctttaacttcaatccgcataaatggctgctggtgaattttgattgtagcgcaatgtgggttaaacgtcgtaccgatctgaccggtgcatttaaactggatccggtgtatctgaaacatagccatcagggtagtggtctgattaccgattatcgtcattggcaactgccgctgggtcgtcgttttcgtagcctgaaaatgtggtttgtgtttcgtatgtatggtgttaaaggactgcaggcctatattcgtaaacatgttcagctgagccatgaatttgaagcatttgtgctgcaggatccgcgttttgaagtttgtgcagaagttaccctgggcctggtttgctttcgtctgaaaggtagtgatggtctgaatgaagccctgctggaacgtattaatagcgcacgtaaaattcatctggttccgtgtcgtctgcgtggtcagtttgttctgcgttttgcaatttgtagccgtaaagttgaaagcggtcatgttcgtctggcatgggaacatattcgtggtctggcagcagagctgctggctgcagaagaaggtaaagcagaaatcaaaagctaa

SEQ ID NO: 18 - protein, 486 aa, Sus scrofa
MNASDFRRRGKEMVDYMADYLEGIEGRQVYPDVQPGYLRPLIPATAPQEPDTFEDILQDVEKIIMPGVTHWHSPYFFAYFPTASSYPAMLADMLCGAIGCIGFSWAASPACTELETVMMDWLGKMLQLPEAFLAGEAGEGGGVIQGSASEATLVALLAARTKVVRRLQAASPGLTQGAVLEKLVAYASDQAHSSVERAGLIGGVKLKAIPSDGKFAMRASALQEALERDKAAGLIPFFVVATLGTTSCCSFDNLLEVGPICHEEDIWLHVDAAYAGSAFICPEFRHLLNGVEFADSFNFNPHKWLLVNFDCSAMWVKRRTDLTGAFKLDPVYLKHSHQGSGLITDYRHWQLPLGRRFRSLKMWFVFRMYGVKGLQAYIRKHVQLSHEFEAFVLQDPRFEVCAEVTLGLVCFRLKGSDGLNEALLERINSARKIHLVPCRLRGQFVLRFAICSRKVESGHVRLAWEHIRGLAAELLAAEEGKAEIKS

SEQ ID NO: 19 - DNA, 1440 bp, Candidatus Koribacter versatilis Ellin345
atgaaccgcatgaaaaacaactttcacatgctgcctgatgattttcgtgcagcaggtcataaagttattgattgggttgcagattatcatgcccacgttgaagattttcgcgttctgagccaggttaaaccgggtgaaatttgtgatggtctgccggatagccctccgcagcagggtgatagcgttaccaatattctgcctgatattgaacgtcatgttctgccaggtattacccattggcagagcccgaacttttatgcatattttccgagcaataattccggtccgagcattctgggtgatctggttagcagcggtctgggtgttcagggtatgctgtgggcaaccagtccggcatgtaccgaagttgaaatgaaaatgctggattggctggttcagatgctgggcctgccggaacattttctgaatagcagcaaacatggtggtggtgttattcaggatagcgcaagcagcgcaaccctgtgtgcactgctggcagcacgtgaacaggcaaccaatggtcagaccaatgaagaaggttgtcgtctgccgctggtttgttataccagcaatcaggcacatagccatgttgagaaagatgttaaagttgcaggtctgggtcgtaaaaatctgcgtctgattgatgtggatcaagaatttgcaatgcgtccggaagcactggaacgtcagattgttgaagataaagcagcaggcaaaatcccgttttttgtttgtgcaaccattggtacaaccagcagcctggcaattgatccgattccggaaattgcagccatttgtaaacgccatggtctgtggctgcatgttgatgcagcaatggcaggcaccgcagcactgtgtccggaatttcgttggacccataatggtgttgaactggcagatagctatgcatttaatccgcacaaatggatgtataccaactttgattgtaccgccttttgggtgaaagatcgtcatgcactgattaatagcctgagcgttgttccggaatatctgcgtaatcaggcaagcgaacagggtgaagtttttgattatcgtgattggcatgttccgctgggtcgtcgttttcgtgccctgaaactgtggtttgtgattcgtcattatggtgtggaaggtctgcagcatcatgttcgtcagaatgttgcatgggcacaagaattcgcagcatgggttaaagcagatagccgttttgaactggttgcaccgcatccgctgagcctggtttgctttcgtctgaaaagcggtgatgcagccagcgaacagctgctgaaacgtgcaaatgaaagcggcaaaatctttatcagccataccaaactggatggcaaatatgttctgcgttttagcattggtcaggcaaaaaccgaacgtcatcatgttgaagccgcatggaaactgattagcgatctggccgatcgtagctaa

SEQ ID NO: 20 - protein, 479 aa, Candidatus Koribacter versatilis Ellin345
MNRMKNNFHMLPDDFRAAGHKVIDWVADYHAHVEDFRVLSQVKPGEICDGLPDSPPQQGDSVTNILPDIERHVLPGITHWQSPNFYAYFPSNNSGPSILGDLVSSGLGVQGMLWATSPACTEVEMKMLDWLVQMLGLPEHFLNSSKHGGGVIQDSASSATLCALLAAREQATNGQTNEEGCRLPLVCYTSNQAHSHVEKDVKVAGLGRKNLRLIDVDQEFAMRPEALERQIVEDKAAGKIPFFVCATIGTTSSLAIDPIPEIAAICKRHGLWLHVDAAMAGTAALCPEFRWTHNGVELADSYAFNPHKWMYTNFDCTAFWVKDRHALINSLSVVPEYLRNQASEQGEVFDYRDWHVPLGRRFRALKLWFVIRHYGVEGLQHHVRQNVAWAQEFAAWVKADSRFELVAPHPLSLVCFRLKSGDAASEQLLKRANESGKIFISHTKLDGKYVLRFSIGQAKTERHHVEAAWKLISDLADRS

SEQ ID NO: 21 - DNA, 1413 bp, Draconibacterium orientale
atgagtccggaacagtttcgcaaagaaggcaaaaaaatcattgattggatcgccgattattacgagcagattgaagattatccggttctgagccaggttaaaccgggtgatattctgaatagcctgccggatgcagcaccgcagcagggtgaaagcattgatgacatgattaaagatctggatgagaaaatcatgcctggcattacccattggcagagcccgaacttttttgcgtattttaacgcaaataccagctttccgagcattctgggtgatctggttagcagcggtctgggtgttcagggtatgctgtgggcaaccagtccggcagcaaccgaagttgaaacccgtgttctggattggctggcagacatgatggatatgcctgaacagttccgtagcaccagcagcggtggtggtgttattcaggataccgcaagtaccgcagcactgaccgcaattattgcagcacgtgaacgtgttaccggctttaaaaccaatgaagatggcctggataaaaaactggttgcatatgttagcagccagacccatagcagcgttgaaaaaggtatcaaaattgcaggtattggtcgtgcaaatctgcgtctgattgatgttgatgaaaattgtagcatgcgtaccgatctgctggaacagcagattaaagaagatcgcgcaaacggtctgattccgttttttgtttgtggcaccattggtacaaccagcagtaccgcaattgatccgctgccggaaattggtcgtattagcaaacaagaaaaatgctggtttcacattgatgcagcaagcctgggcaccgcaatgctgtgtccggaatttcgtcatctggcagatggtgttgaactggcagatagctatagttttaatccgcacaaatggatgttcaccaactttgattgcaatgttttttgggttgccaatcgcagcgaactgattaatacctttagcattctgccggaatatctgcgtaataaagccaccgaaagcggtgaagtttttgattatcgtgattggcatattcagctgggtcgtcgttttcgtgcactgaaactgtggtttgttattcgtcattatggtgtggaaggtctgcagcatctgattcatgaacatgtgcgtctggcaaaagcatttgaaaatcgtgttaacgatagcgaggattttgaagttgttgttccgaccaccatgaatctggtttgctttcgccataaaagtgatgatgcctttaacctgaaactgatgaacaccattaatgaagcgggtaaaatcttttttacccacaccaaaattaacggtcaggttgttctgcgcatgaatattggtcagacacatagcgaagaaaaacatgtgaataacgcctggaaaatcattcaagaaaccgcaaaaaccctggaataa

SEQ ID NO: 22 - protein, 470 aa, Draconibacterium orientale
MSPEQFRKEGKKIIDWIADYYEQIEDYPVLSQVKPGDILNSLPDAAPQQGESIDDMIKDLDEKIMPGITHWQSPNFFAYFNANTSFPSILGDLVSSGLGVQGMLWATSPAATEVETRVLDWLADMMDMPEQFRSTSSGGGVIQDTASTAALTAIIAARERVTGFKTNEDGLDKKLVAYVSSQTHSSVEKGIKIAGIGRANLRLIDVDENCSMRTDLLEQQIKEDRANGLIPFFVCGTIGTTSSTAIDPLPEIGRISKQEKCWFHIDAASLGTAMLCPEFRHLADGVELADSYSFNPHKWMFTNFDCNVFWVANRSELINTFSILPEYLRNKATESGEVFDYRDWHIQLGRRFRALKLWFVIRHYGVEGLQHLIHEHVRLAKAFENRVNDSEDFEVVVPTTMNLVCFRHKSDDAFNLKLMNTINEAGKIFFTHTKINGQVVLRMNIGQTHSEEKHVNNAWKIIQETAKTLE

SEQ ID NO: 23 - DNA, 1863 bp, Enterococcus faecalis
atgaaaaacgaaaaattagcaaaaggcgaaatgaaccttaatgcactattcattggggacaaagccgaaaacggacaattatataaagacttgttgatcgacttagtagatgaacatttaggatggcgtcaaaactacatgccacaagacatgccagttatctcttctcaagaacgcacatctgaaagctacgaaaaaactgtcaaccatatgaaagatgtattgaatgaaatctcttctcgcatgcgtacacattcagttccatggcatacagcaggtagatattggggacatatgaactcagaaacattaatgccttctctattagcttacaactttgcaatgctatggaacgggaacaacgttgcctatgaatcttctccagcaacttctcaaatggaagaagaagtaggacatgaatttgctcacttgatgagctacaaaaatggttggggacacatcgttgctgatggttctttagctaacttagaaggcttatggtatgcccgtaacattaaatcattaccatttgctatgaaagaagtaaaaccagaattagttgctggcaaatcagattgggaactattgaacatgccaacaaaagaaattatggacttattagaatcagctgaagatgaaattgatgaaatcaaagctcattcagctcgttcaggtaaacatttacaagcaatcggtaaatggttagttccacaaactaaacactattcttggttaaaagctgctgatattatcggtatcggtttagatcaagttatcccagtaccagttgaccataactaccgtatggatatcaacgaattagaaaaaatcgttcgtggcttagcagaagaacaaattccagtgctaggtgttgtcggtgtagttggttctactgaagaaggtgccgttgactcaatcgataaaattattgctttacgcgatgaattaatgaaagacggtatttactattatgtacacgtggatgctgcttatggtggttatggacgtgccatcttcttagacgaagacaacaacttcatcccttacgaagatttacaagatgttcacgaagaatacggtgtcttcaaagagaaaaaagaacacatttcaagagaagtgtatgatgcatataaagcaatcgaattagcagaatcagtaacaattgaccctcataaaatgggttatatcccttattcagctggtggtatcgttatccaagatatccgtatgcgcgacgttatttcttacttcgcaacttacgtatttgaaaaaggtgccgatattcctgcgttactaggtgcttatatccttgaaggttcaaaagctggtgcaacagctgcttctgtatgggctgcacatcacgttttacctttaaacgttgcaggttatggtaaattaattggtgcttcaattgaaggctcacatcatttctataacttcttgaatgatttaacattcaaagttggcgacaaagaaatcgaagtccacacattaactcatccagacttcaacatggttgactatgtgtttaaagaaaaaggcaacgatgatttagtagcaatgaacaaattaaaccatgatgtatatgactatgcatcatacgttaaaggaaatatttacaacaacgaattcattacttcacatactgactttgctattccagattatggcaacagcccattaaaatttgtaaacagcttaggatttagtgacgaagaatggaaccgtgcaggtaaagtaactgtgttacgtgctgctgtaatgactccatacatgaacgataaagaagaatttgatgtttacgctcctaagattcaagctgctttacaagaaaaattagaacaaatttacgacgtaaaataa

SEQ ID NO: 24 - DNA, 1863 bp, Enterococcus faecalis
atgaaaaatgagaagttggcgaagggggaaatgaatctgaatgcattgttcatcggagacaaggctgagaatggccagttatacaaggatcttttgattgacttggttgatgagcaccttgggtggcgtcagaattacatgcctcaggatatgcccgtaattagtagtcaggaacgtactagcgagtcgtacgaaaagactgttaaccatatgaaggacgtcctgaacgaaatctccagccgcatgcgcacgcacagcgtaccgtggcataccgcgggccgctactggggtcacatgaactcagagacccttatgccttccctgcttgcctataattttgcaatgctttggaatgggaacaatgttgcttatgaatcatctccagctacctctcagatggaggaggaggtaggtcacgagtttgctcatctgatgtcatacaaaaatgggtggggacatatcgtcgcggacggcagtttagcaaacctggagggactttggtatgctcgtaatatcaaaagccttcccttcgctatgaaggaggttaagcccgaattagttgccggcaagtccgactgggaactgcttaatatgcctacaaaggaaattatggatctgttagaaagtgcagaagacgagatcgacgaaattaaagcacattctgcacgctcaggcaaacacttgcaagctattggtaaatggctggtgccccaaacaaaacactatagttggttaaaggctgcggacatcatcggtatcggcctggatcaggtgatccccgtgcccgtggaccataattatcgcatggacattaatgaattggaaaaaatcgtacgcggcttagctgaagagcagatcccagttttaggcgtggtaggggtagtaggcagcaccgaagagggagccgttgactctatcgacaaaatcatcgcgttacgcgacgagctgatgaaagatggtatttattactatgtccatgtagacgcagcctatgggggatatggacgtgcaatttttttagatgaggacaataacttcatcccctatgaggatctgcaagacgtccatgaagaatatggcgtgtttaaagaaaaaaaagagcacatttctcgtgaagtatacgatgcctacaaagccatcgagcttgccgagagcgttaccattgacccgcacaagatgggctatatcccgtatagtgctggcggcattgtcattcaggatattcgcatgcgtgatgtcatctcatatttcgcaacctacgtgtttgagaaaggcgcggatatccctgcactgttaggagcctacatcttagaggggtccaaagctggcgcaacggccgcttcagtctgggctgcacatcacgtacttccgctgaacgtagcaggttacggtaaattaatcggtgcttccatcgaagggtcgcaccatttttataatttcttaaatgatctgacatttaaagtaggagacaaagaaattgaggttcacaccttgacgcacccagattttaatatggtagactacgtattcaaagagaagggaaatgacgatttggttgcaatgaataagctgaaccatgacgtatacgactatgcttcttacgttaagggtaatatctataataacgagtttattacgtctcacaccgacttcgcaattcccgactacggaaactcgccgctgaagttcgtaaatagtctggggtttagtgatgaggagtggaatcgtgctggaaaggtaactgtattgcgcgccgccgtcatgaccccatatatgaacgacaaagaggagttcgacgtatatgctcctaagattcaggctgctcttcaagagaaattagaacagatttatgatgtcaaatga

SEQ ID NO: 25 - protein, 620 aa, Enterococcus faecalis
MKNEKLAKGEMNLNALFIGDKAENGQLYKDLLIDLVDEHLGWRQNYMPQDMPVISSQERTSESYEKTVNHMKDVLNEISSRMRTHSVPWHTAGRYWGHMNSETLMPSLLAYNFAMLWNGNNVAYESSPATSQMEEEVGHEFAHLMSYKNGWGHIVADGSLANLEGLWYARNIKSLPFAMKEVKPELVAGKSDWELLNMPTKEIMDLLESAEDEIDEIKAHSARSGKHLQAIGKWLVPQTKHYSWLKAADIIGIGLDQVIPVPVDHNYRMDINELEKIVRGLAEEQIPVLGVVGVVGSTEEGAVDSIDKIIALRDELMKDGIYYYVHVDAAYGGYGRAIFLDEDNNFIPYEDLQDVHEEYGVFKEKKEHISREVYDAYKAIELAESVTIDPHKMGYIPYSAGGIVIQDIRMRDVISYFATYVFEKGADIPALLGAYILEGSKAGATAASVWAAHHVLPLNVAGYGKLIGASIEGSHHFYNFLNDLTFKVGDKEIEVHTLTHPDFNMVDYVFKEKGNDDLVAMNKLNHDVYDYASYVKGNIYNNEFITSHTDFAIPDYGNSPLKFVNSLGFSDEEWNRAGKVTVLRAAVMTPYMNDKEEFDVYAPKIQAALQEKLEQIYDVK

SEQ ID NO: 26 - DNA, 132 bp, Artificial Sequence
  Hiracin JM79
gcgacttattacggcaacgggttgtattgcaataaagaaaagtgttgggtggactggaatcaggcgaaaggggaaatcggaaaaatcattgttaatggatgggtcaatcacggaccgtgggcaccacgtcgc

SEQ ID NO: 27 - protein, 65 aa, Artificial Sequence
  Hiracin JM79
MKKTAIAIAVALAGFATVAQAATYYGNGLYCNKEKCWVDWNQAKGEIGKIIVNGWVNHGPWAPRR

SEQ ID NO: 28 - DNA, 147 bp, Artificial Sequence
  Ubericin A
aaaacagtaaattatggaaacgggctttattgtaaccaaaaaaagtgctgggttaattggtcagagaccgcgactactattgtaaacaactccattatgaacgggttgactggtggaaatgccgggtggcattccggtggccgtgca

SEQ ID NO: 29 - protein, 70 aa, Artificial Sequence
  Ubericin A
MKKTAIAIAVALAGFATVAQAKTVNYGNGLYCNQKKCWVNWSETATTIVNNSIMNGLTGGNAGWHSGGRA

SEQ ID NO: 30 - DNA, 141 bp, Artificial Sequence
  Enterocin A
acgacacactccggtaaatattacggtaacggcgtttactgcacaaagaataaatgcactgtagattgggccaaggcaacgacctgcattgctgggatgtctattggcggatttctgggtggggcaatccccggcaaatgt

SEQ ID NO: 31 - protein, 68 aa, Artificial Sequence
  Enterocin A
MKKTAIAIAVALAGFATVAQATTHSGKYYGNGVYCTKNKCTVDWAKATTCIAGMSIGGFLGGAIPGKC

SEQ ID NO: 32 - DNA, 36 bp, Artificial Sequence
  MSKL 7 promoter
tgcttgactcgtcgttcctcctacgtgtataattgg

SEQ ID NO: 33 - DNA, 36 bp, Artificial Sequence
  MSKL 8 promoter
tgcttgactcgtcgttatcctacgtgtataattggc

SEQ ID NO: 34 - DNA, 30 bp, Artificial Sequence
  Trc promoter
ttgacaattaatcatccggctcgtataatg

SEQ ID NO: 35 - DNA, 72 bp, Artificial Sequence
  Trc promoter with lac operator (with lacl binding site)
ttgacaattaatcatccggctcgtataatgtgtggaattgtgagcggataacaatttcacacaggagtaaaa

SEQ ID NO: 36 - DNA, 55 bp, Artificial Sequence
  Trc promoter with lac operator (without lacl binding site)
ttgacaattaatcatccggctcgtataatgtgtggaatttcacacaggagtaaaa

SEQ ID NO: 37 - DNA, 35 bp, Artificial Sequence
  BBa_J23100
ttgacggctagctcagtcctaggtacagtgctagc

SEQ ID NO: 38 - DNA, 35 bp, Artificial Sequence
  J23102
ttgacagctagctcagtcctaggtactgtgctagc

SEQ ID NO: 39 - DNA, 35 bp, Artificial Sequence
  synthetic promoter
tttacagctagctcagtcctaggtattatgctagc

SEQ ID NO: 40 - DNA, 35 bp, Artificial Sequence
  synthetic promoter
tttacggctagctcagtcctaggtactatgctagc

SEQ ID NO: 41 - DNA, 35 bp, Artificial Sequence
  synthetic promoter
tttacggctagctcagtcctaggtatagtgctagc

SEQ ID NO: 42 - DNA, 35 bp, Artificial Sequence
  synthetic promoter
tttacggctagctcagccctaggtattatgctagc

SEQ ID NO: 43 - DNA, 35 bp, Artificial Sequence
  synthetic promoter
ctgacagctagctcagtcctaggtataatgctagc

SEQ ID NO: 44 - DNA, 35 bp, Artificial Sequence
  synthetic promoter
tttacggctagctcagtcctaggtacaatgctagc

SEQ ID NO: 45 - DNA, 35 bp, Artificial Sequence
  synthetic promoter
ttgacggctagctcagtcctaggtatagtgctagc

SEQ ID NO: 46 - DNA, 35 bp, Artificial Sequence
  synthetic promoter
tttatggctagctcagtcctaggtacaatgctagc

SEQ ID NO: 47 - DNA, 35 bp, Artificial Sequence
  synthetic promoter
tttatagctagctcagcccttggtacaatgctagc

SEQ ID NO: 48 - DNA, 35 bp, Artificial Sequence
  synthetic promoter
ttgacagctagctcagtcctagggactatgctagc

SEQ ID NO: 49 - DNA, 35 bp, Artificial Sequence
  synthetic promoter
ttgacagctagctcagtcctagggattgtgctagc

SEQ ID NO: 50 - DNA, 35 bp, Artificial Sequence
  synthetic promoter
ttgacggctagctcagtcctaggtattgtgctagc

SEQ ID NO: 51 - DNA, 669 bp, Artificial Sequence
  folE codon optimized
atgccttctctttccaaggaggcggcacttgttcacgaagctttggtggcgcgcgggttagaaacgccactgcgcccacccgttcacgaaatggacaatgagacccgcaagagtcttatcgcggggcatatgacagaaattatgcagttgttaaacttagacttggctgacgactccctgatggagacccctcatcgtattgccaagatgtacgttgatgagatttttagcggcttagactatgcaaattttcccaaaattactctgattgagaataagatgaaagtggacgagatggtgacagttcgtgatattacacttacaagtacgtgtgagcatcacttcgttacaatcgacggcaaggccacggtcgcctacatcccaaaagatagtgtgatcggccttagcaaaatcaaccgcattgttcaattcttcgcgcagcgtccacaagtgcaagaacgcttgactcaacaaatcctgattgcgttacagacattacttggcacaaacaatgtggcagtatctattgatgcagtgcactactgtgtgaaagctcgtggcattcgtgatgccacatccgcgacgaccacaatctccctgggcggccttttcaagtccagccagaacacgcgtcatgaattcttacgtgcggtacgccaccataactga

SEQ ID NO: 52 - protein, 222 aa, Artificial Sequence
  folE codon optimized protein sequence
MPSLSKEAALVHEALVARGLETPLRPPVHEMDNETRKSLIAGHMTEIMQLLNLDLADDSLMETPHRIAKMYVDEIFSGLDYANFPKITLIENKMKVDEMVTVRDITLTSTCEHHFVTIDGKATVAYIPKDSVIGLSKINRIVQFFAQRPQVQERLTQQILIALQTLLGTNNVAVSIDAVHYCVKARGIRDATSATTTISLGGLFKSSQNTRHEFLRAVRHHN

SEQ ID NO: 53 - DNA, 723 bp, Artificial Sequence
  folM codon optimized
atgggtaagacccaatctttgcctattttaatcactgggggcggccgccgtatcggcctggcattggcgtggcactttattaaccaaaagcagcccgtaatcgtctcatatcgcacgcactacccagccattgacgggttgattaaagctggtgcacagtgtatccaggcagatttcagcactaacgatggggtgatggcttttgcagacgaagtgctgaagtcgacacatggcttgcgtgctatcttacacaacgcgtctgcatggatggcagagaaaccgggcgcacccttaacggacgtgttagcgtgtatgatgcaaatccatgtaaatactccatatttactgaatcacgcacttgaacgtttactgcgcggtcacggccacgcagcttccgacatcattcatttcacagactacgttgtcgaacgcggaagcgataaacacatcgcttatgctgccagtaaagcggcacttgataacatgacccgttcctttgcacgcaagcttgcgccagaagtcaaggtgaactcgatcgcgcctagtctgatccttttcaacgagcacgacgatgcggaatatcgtcagcaagctctgaataagagcttgatgaagacggctccgggtgagaaagaggtaatcgaccttgtcgactacctgcttacaagctgtttcgtgacgggtcgctctctgcctcttgatggtggccgtcatcttcgctga

SEQ ID NO: 54 - protein, 240 aa, Artificial Sequence; identical to SEQ ID NO: 12
  folM codon optimized protein sequence

SEQ ID NO: 55 - DNA, 36 bp, Artificial Sequence
  MSKL consenus sequence
  misc_feature (1)..(1): d is a or g or t
  misc_feature (2)..(2): s is g or c
  misc_feature (3)..(3): n is a, c, g, or t
  misc_feature (4)..(4): y is c or t
  misc_feature (5)..(5): k is g or t
  misc_feature (6)..(6): n is a, c, g, or t
  misc_feature (7)..(7): r is a or g
  misc_feature (8)..(8): y is c or t
  misc_feature (9)..(9): d is a or g or t
  misc_feature (10)..(10): m is a or c
  misc_feature (11)..(11): d is a or g or t
  misc_feature (12)..(12): h is a or c or t
  misc_feature (13)..(13): b is c or g or t
  misc_feature (14)..(14): r is a or g
  misc_feature (15)..(15): n is a, c, g, or t
  misc_feature (16)..(16): d is a or g or t
  misc_feature (17)..(17): h is a or c or t
  misc_feature (18)..(18): y is c or t
  misc_feature (19)..(19): b is c or g or t
  misc_feature (20)..(20): v is a or c or g
  misc_feature (21)..(21): h is a or c or t
  misc_feature (22)..(22): n is a, c, g, or t
  misc_feature (23)..(23): h is a or c or t
  misc_feature (24)..(24): n is a, c, g, or t
  misc_feature (25)..(25): b is c or g or t
  misc_feature (26)..(26): n is a, c, g, or t
  misc_feature (27)..(30): d is a or g or t
  misc_feature (31)..(31): n is a, c, g, or t
  misc_feature (32)..(32): h is a or c or t
  misc_feature (33)..(33): k is g or t
  misc_feature (34)..(34): d is a or g or t
  misc_feature (35)..(36): n is a, c, g, or t
dsnyknrydmdhbrndhybvhnhnbnddddnhkdnn

SEQ ID NO: 56 - DNA, 35 bp, Artificial Sequence
  Anderson consensus sequence
  misc_feature (1)..(1): y is c or t
  misc_feature (3)..(3): k is g or t
  misc_feature (5)..(5): y is c or t
  misc_feature (6)..(6): r is a or g
  misc_feature (17)..(17): y is c or t
  misc_feature (21)..(21): w is a or t
  misc_feature (24)..(24): k is g or t
  misc_feature (26)..(26): y is c or t
  misc_feature (28)..(28): r is a or g
ytkayrgctagctcagycctwggkaywrtgctagc